(19) United States
(12) Patent Application Publication
(10) Pub. No.: US 2020/0061117 A1
(43) Pub. Date: Feb. 27, 2020

(54) Protective Chimeric Antigen Receptor Stem Cell Gene Therapy for Viral Infection

(71) Applicant: The Regents of the University of California, Oakland, CA (US)

(72) Inventors: Scott G. Kitchen, Los Angeles, CA (US); Anjie Zhen, Los Angeles, CA (US); Jerome A. Zack, Tarzana, CA (US); Masakazu Kamata, Los Angeles, CA (US); Irvin Chen, Palos Verdes Estates, CA (US)

(21) Appl. No.: 16/611,797

(22) PCT Filed: May 4, 2018
(86) PCT No.: PCT/US2018/031145

§ 371 Date: Nov. 7, 2019

Related U.S. Application Data
(60) Provisional application No. 62/503,203, filed on May 8, 2017.

(51) Int. Cl. A61K 35/17; A61K 39/12; A61P 31/18; C07K 14/725; C07K 14/73; C12N 15/86; C07K 14/16

(52) U.S. Cl. CPC A61K 35/17; A61K 39/12; A61P 31/18; A61K 2039/5156; C07K 14/70514; C12N 15/86; C07K 14/162; C07K 14/7051

(57) ABSTRACT

Disclosed herein is a CAR construct encoding an inhibitor of immunodeficiency virus fusion and human CD4 extracellular and transmembrane domains linked to a human CD3ζ signaling domain, and methods of making and using thereof to treat, reduce, or inhibit HIV in subjects.

### CROSS-REFERENCE TO RELATED APPLICATIONS

This application claims the benefit of U.S. Application No. 62/503,203, filed May 8, 2017, which is herein incorporated by reference in its entirety.

### ACKNOWLEDGEMENT OF GOVERNMENT SUPPORT

This invention was made with Government support under AI117941, awarded by the National Institutes of Health. The Government has certain rights in the invention.

### BACKGROUND OF THE INVENTION

### 1. Field of the Invention

The present invention generally relates to recombinant and engineered cells expressing a chimeric antigen receptor (CAR) specific for an immunodeficiency virus such as HIV or an epitope thereof

### 2. Description of the Related Art

The seminal case study for HIV cure/remission is the Berlin patient, who received an allogeneic, HLA-matched hematopoietic stem and progenitor cell (HSPC) transplant from a donor homozygous for CCR5432 (23), and has stimulated the search for HSPC-based cure approaches. Allogeneic HSPC transplantation without CCR5432-protected donor cells in 2 HIV⁺ recipients initially resulted in undetectable HIV-1 after patients achieved full donor chimerism; this was likely due to a “graft versus reservoir” effect in which donor lymphocytes destroyed latently infected host cells. Ultimately, this intervention failed to eradicate latently infected cells, which rebounded after cART cessation (24). These studies indicated that a combinatorial approach, rendering the blood and immune system resistant to infection and at the same time harnessing the immune system to attack infected cells, would be required. Numerous studies have used various gene therapy and gene editing approaches to genetically modify autologous stem cells, rendering them resistant to HIV infection (13, 14, 25-29).

### SUMMARY OF THE INVENTION

In some embodiments, the present invention is an expression vector as described herein. In some embodiments, the expression vector comprises a nucleic acid sequence encoding an inhibitor of immunodeficiency virus fusion and human CD4 extracellular and transmembrane domains linked to a human CD3ζ signaling domain. In some embodiments, the inhibitor of immunodeficiency virus fusion is C46. In some embodiments, expression of the inhibitor of immunodeficiency virus fusion is regulated by a ubiquitin C promoter. In some embodiments, expression of the human CD4 extracellular and transmembrane domains linked to a human CD3ζ signaling domain is regulated by an EF-1 alpha promoter. In some embodiments, expression of the inhibitor of immunodeficiency virus fusion is regulated by a ubiquitin C promoter and expression of the human CD4 extracellular and transmembrane domains linked to a human CD3ζ signaling domain is regulated by an EF-1 alpha promoter. In some embodiments, the expression vector comprises a nucleic acid sequence encoding an inhibitor of immunodeficiency virus fusion under a ubiquitin C promoter, and human CD4 extracellular and transmembrane domains linked to a human CD3ζ signaling domain under an EF-1 alpha promoter. In some embodiments, the human CD4 extracellular domain is truncated, e.g., comprises only one, two, or three of the D1-D4 domains. In some embodiments, the expression vector is a lentiviral vector. In some embodiments, the expression vector has an FG11 lentivirus vector backbone. In some embodiments, the expression vector has an FG12 lentivirus vector backbone. In some embodiments, the expression vector has an SFFV lentivirus vector backbone.

In some embodiments, the present invention is a host cell comprising an expression vector as described herein. In some embodiments, the expression vector comprising a nucleic acid sequence encoding an inhibitor of immunodeficiency virus fusion and human CD4 extracellular and transmembrane domains linked to a human CD3ζ signaling domain. In some embodiments, the inhibitor of immunodeficiency virus fusion is C46. In some embodiments, expression of the inhibitor of immunodeficiency virus fusion is regulated by a ubiquitin C promoter. In some embodiments, expression of the human CD4 extracellular and transmembrane domains linked to a human CD3ζ signaling domain is regulated by an EF-1 alpha promoter. In some embodiments, expression of the inhibitor of immunodeficiency virus fusion is regulated by a ubiquitin C promoter and expression of the human CD4 extracellular and transmembrane domains linked to a human CD3ζ signaling domain is regulated by an EF-1 alpha promoter. In some embodiments, the human CD4 extracellular domain is truncated, e.g., comprises only one, two, or three of the D1-D4 domains. In some embodiments, the expression vector is a lentiviral vector. In some embodiments, the expression vector has an FG11 lentivirus vector backbone. In some embodiments, the expression vector has an FG12 lentivirus vector backbone. In some embodiments, the expression vector has an SFFV lentivirus vector backbone. In some embodiments, the host cell is a hematopoietic stem cell or a hematopoietic progenitor cell. In some embodiments, the host cell is a T cell, an NK cell, a B cell, or a tissue cell. In some embodiments, the host cell expresses a chimeric antigen receptor encoded by an expression vector as described herein. In some embodiments, the present invention is a cell that is the progeny of a host cell as described herein. In some embodiments, the cell expresses a chimeric antigen receptor encoded by an expression vector as described herein. In some embodiments, the cell is a T cell, an NK cell, a B cell, or a tissue cell.

In some embodiments, the present invention is a method of treating, reducing, or inhibiting an infection by an immunodeficiency virus in a subject, which comprises administering one or more expression vectors as described herein, e.g., paragraph [0011], to the subject. In some embodiments, the method further comprises administering to the subject a therapeutically effective amount of an antiretroviral therapeutic. In some embodiments, the method further comprises administering to the subject an effective amount of one or more latency reversing agents. In some embodiments, the method further comprises expanding the amount of cells expressing the chimeric antigen receptor in the subject by administering to the subject an effective amount of an agent that binds human CD4. In some embodiments, the agent is one or more antigens that bind human CD4. In some embodiments, the one or more antigens is gp120 or an epitope thereof. In some embodiments, the agent is an HIV vaccine that acts by binding human CD4. In some embodiments, the subject is human. In some embodiments, the immunodeficiency virus is a human immunodeficiency virus. In some embodiments, the immunodeficiency virus is a simian immunodeficiency virus having an envelope of the human immunodeficiency virus. In some embodiments, the human immunodeficiency virus is HIV-1 or HIV-2.

In some embodiments, the present invention is a method of treating, reducing, or inhibiting an infection by an immunodeficiency virus in a subject, which comprises transplanting one or more cells as described herein, e.g., paragraph [0012], in the subject. In some embodiments, the method further comprises administering to the subject a therapeutically effective amount of an antiretroviral therapeutic. In some embodiments, the method further comprises administering to the subject an effective amount of one or more latency reversing agents. In some embodiments, the method further comprises expanding the amount of cells expressing the chimeric antigen receptor in the subject by administering to the subject an effective amount of an agent that binds human CD4. In some embodiments, the agent is one or more antigens that bind human CD4. In some embodiments, the one or more antigens is gp120 or an epitope thereof. In some embodiments, the agent is an HIV vaccine that acts by binding human CD4. In some embodiments, the subject is human. In some embodiments, the immunodeficiency virus is a human immunodeficiency virus. In some embodiments, the immunodeficiency virus is a simian immunodeficiency virus having an envelope of the human immunodeficiency virus. In some embodiments, the human immunodeficiency virus is HIV-1 or HIV-2.

### DESCRIPTION OF THE DRAWINGS

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

This invention is further understood by reference to the drawings wherein:

FIG. 1, FIG. 2, and FIG. 3 show that C46CD4CAR cells resist HIV infection and respond to cognate antigen. FIG. 1 schematically shows the lentiviral vectors exemplified herein. FIG. 2 shows expression levels. Jurkat cells were mock transduced, or transduced with CD4CAR or C46CD4CAR, then infected with HIV and cultured for 3 days. Intracellular expression of HIV-1 Gag was measured by flow cytometry using KC57 antibody. FIG. 3: Pigtail macaque T cells were activated and transduced with either C46CD4CARΔzeta (control) or C46CD4CAR. 2 days following transduction, cells were stimulated with either uninfected control T1 cells or HIV-infected T1 cells. Intracellular cytokines were measured by flow analysis.

FIG. 4, FIG. 5, FIG. 6, and FIG. 7 show engraftment of HSPC-based C46CAR cells is multilineage. FIG. 4: Lentivirus gene marking in transplanted subjects following autologous transplantation, measured by Taqman. FIG. 5: Detection of HSPC-based C46CAR cells by flow cytometry with anti-human CD4 antibody clone 13B8.2, which does not detect Macaca nemestrina CD4. FIG. 6: Percent of cells expressing huCD4⁺ among total peripheral PBMCs, at approximately 28 days post-transplant. FIG. 7: Multilineage engraftment of lentivirus-modified cells in peripheral blood was measured from control subjects and C46 subjects. One representative control subject (Control 2) and C46 subject (CAR 2) are shown.

FIG. 8, FIG. 9, and FIG. 10 show reduced viral rebound in C46 subjects. FIG. 8: C46 subjects and control subjects were challenged with SHIV-C via the intravenous route. Approximately 24 weeks later, cART was initiated and administered for 28 weeks. Following cART withdrawal, subjects were monitored for approximately 15 weeks prior to necropsy. FIG. 9: Plasma viral load was monitored longitudinally after SHIV challenge. Dotted line indicates limit of detection (LOD) of 30 copies/mL. FIG. 10: Log reduction of plasma viremia following post-cART viral rebound, relative to comparable time points during primary infection.

FIG. 11, FIG. 12, FIG. 13, FIG. 14, and FIG. 15 show cells expressing C46CD4CAR expand in response to SHIV antigen in vivo. FIG. 11: Lentiviral gene marking was measured by Taqman from peripheral blood at the indicated time points from C46CD4CAR (solid lines) and C46CD4CARΔzeta transplanted subjects (dashed lines). FIG. 12-FIG. 15: Viral load (left Y axis) and percentage of cells in PBMCs expressing CARs (right Y axis) were measured longitudinally from peripheral blood from Control 1 (FIG. 12), Control 2 (FIG. 13), CAR 1 (FIG. 14), and CAR 2 (FIG. 15) subjects.

FIG. 16, FIG. 17, FIG. 18, FIG. 19, and FIG. 20 show T cells expressing C46CD4CAR develop into effector cells in response to viral replication. FIG. 16: Percentages of unmarked (CAR⁻) naïve, memory, and effector T cells and C46CD4CAR⁺ T cells from peripheral blood from one representative subject, collected at the indicated time points. FIG. 17-FIG. 20: Quantitation of naïve, memory, and effector subsets from unmarked T cells and gene modified T cells in peripheral blood from Control 1 (FIG. 17), Control 2 (FIG. 18), CAR 1 (FIG. 19) and CAR 2 (FIG. 20) subjects.

FIG. 21, FIG. 22, and FIG. 23 show that C46CD4CAR protects CD4⁺ T-cells and decrease viral load in multiple tissues. FIG. 21, Panels A-C: GI biopsies were collected from colon and duodenum/jejunum from C46 subjects and control subjects prior to SHIV infection (average of all 4 subjects) and after infection and cART withdrawal (values for C46 subjects and control subjects are shown separately). Shown are CD4/8 ratio (FIG. 21, Panel A), % CD4⁺ T cells (FIG. 21, Panel B), and % CD4⁺ effector memory T-cells (FIG. 21, Panel C). FIG. 22: At necropsy, Taqman was used to measure lentivirus gene marking from lymphoid tissues (spleen and mesenteric, axillary, inguinal, and submandibular lymph nodes), gastrointestinal tract (duodenum, jejunum, ileum, cecum, colon, rectum) and brain tissues (hippocampus, basal ganglia, thalamus, parietal cortex, cerebellum). FIG. 23: Normalized SHIV RNA copy number from same tissues as in FIG. 22. *p<0.01, **p<0.001, ***p<0.0001 by Mann-Whitney test.

FIG. 24 are bar graphs summarizing lentiviral gene marking in transduced HSPC infusion products. Four male juvenile pigtailed macaques were transplanted with autologous HSPCs transduced with lentiviruses expressing C46CD4CAR (CAR) or C46CD4CARΔzeta (Control). Colony forming assays were plated from a small aliquot of transduced CD34⁺ cells that were infused into each autologous recipient. “Percent Lenti⁺ Colonies” represents the number of lentivirus positive colonies divided by actin-positive colonies, measured by PCR; numerical values are displayed over each bar.

FIG. 25 shows hematopoietic recovery following transplant with HSPC-based CAR cells. Four male juvenile pigtailed macaques were transplanted with autologous HSPC-based C46CAR cells (gray and black lines) or HSPC-based CAR cells expressing C46CD4CARΔzeta (gray and black dashed lines). (Panel A) Total white blood cell, (Panel B) Platelet, (Panel C) Neutrophil, and (Panel D) Lymphocyte values were measured by automated differential count. Dotted straight lines represent normal values.

FIG. 26 summarizes surface phenotyping of cells expressing CARs in tissues. At necropsy, the indicated tissues were collected from C46 subjects (Panels A-B) and control subjects (Panels C-D). Flow cytometry was used to determine the percentage of CD45⁺ cells that were CD4⁺ or CD8⁺ T cells, CD20⁺ B cells, CD14⁺ macrophages/monocytes, and CD2⁺ NKG2a⁺ NK cells. In the bar graphs, the cells directly above the CD8+ T-cells are CD20+B cells and the cells directly below the CD8+ T-cells are CD4+ T-cells.

FIG. 27 graphically summarizes normalized SHIV RNA copies from multiple tissues. Individual values are shown from C46 subjects and control subjects, as analyzed in FIG. 23.

### DETAILED DESCRIPTION OF THE INVENTION

As disclosed herein, engineered cells expressing chimeric antigen receptors (CARs) specific against immunodeficiency virus (SHIV and HIV) having an HIV envelope exhibited significant antiviral in vivo efficacy even at low levels. Specifically, HSPC-based CAR cells expressing a protective CD4 chimeric antigen receptor (C46CD4CAR) were used to engineer T-cells against cells infected with a simian immunodeficiency virus having an HIV envelope (SHIV) in pigtail macaques. The engineered cells were generated from hematopoietic stem and progenitor cells (HSPCs). As used herein, “HSPC-based CAR cells” refer to a cell engineered to express a CAR by transducing a HSPC with a CAR construct and progeny thereof. As used herein, “HSPC-based C46CAR cells” refer to HSPC-based CAR cells that express C46CD4CAR. As used herein, “HSPC” refers to a hematopoietic stem cell (HSC) and/or a hematopoietic progenitor cell (HPC). As used herein, a “CAR construct” refers to an expression vector designed to be capable of expression of a given CAR construct, such as C46CD4CAR, in a cell when provided therein.

The HSPC-based C46CAR cells persisted for more than 2 years without any measurable toxicity and were capable of multilineage engraftment. Levels of the HSPC-based CAR cells in the periphery corresponded closely to the levels of viral antigen. Treatment with combination antiretroviral therapy (cART) followed by discontinuation of cART resulted in lower viral rebound in subjects treated with the HSPC-based CAR cells relative to controls, and demonstrated an immune memory-like response. The HSPC-based C46CAR cells were found in multiple lymphoid tissues. Treatment with the HSPC-based CAR cells resulted in decreased tissue-associated SHIV RNA levels and substantially higher CD4/CD8 ratios in the gut as compared to controls.

Exemplified CAR Construct

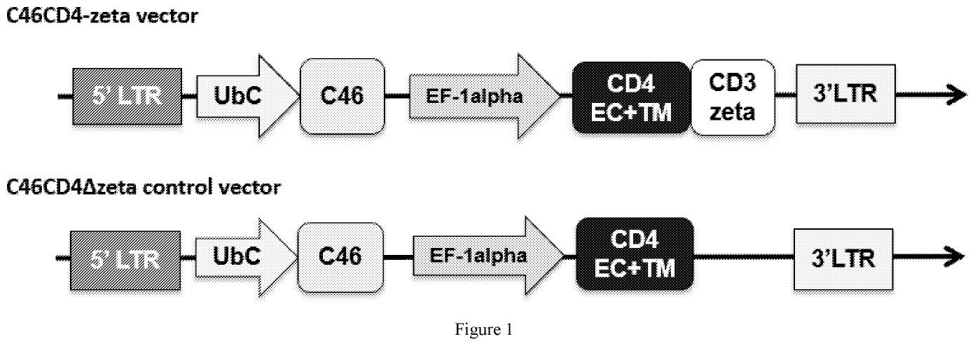

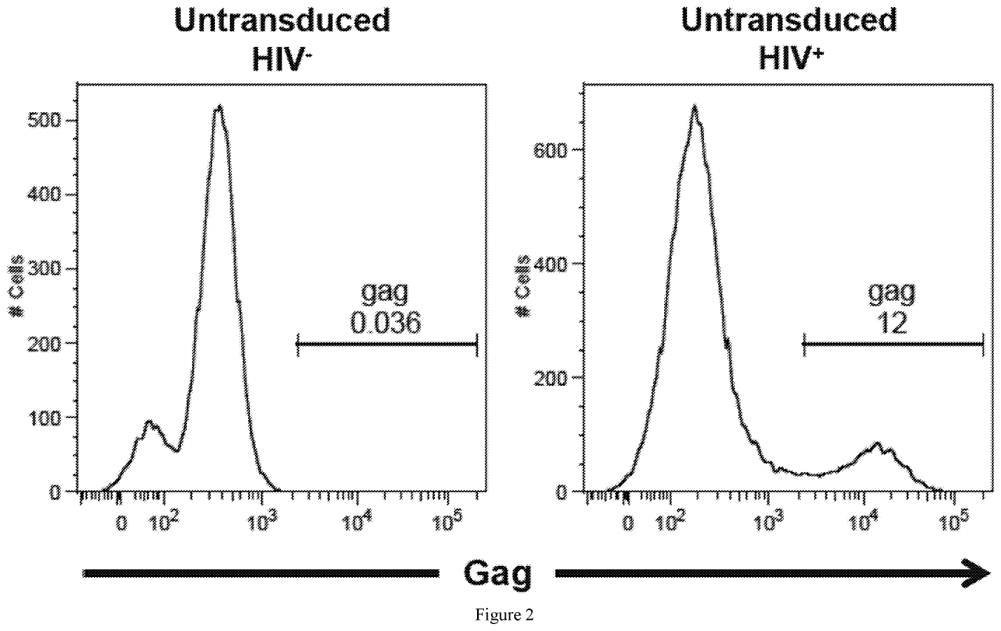

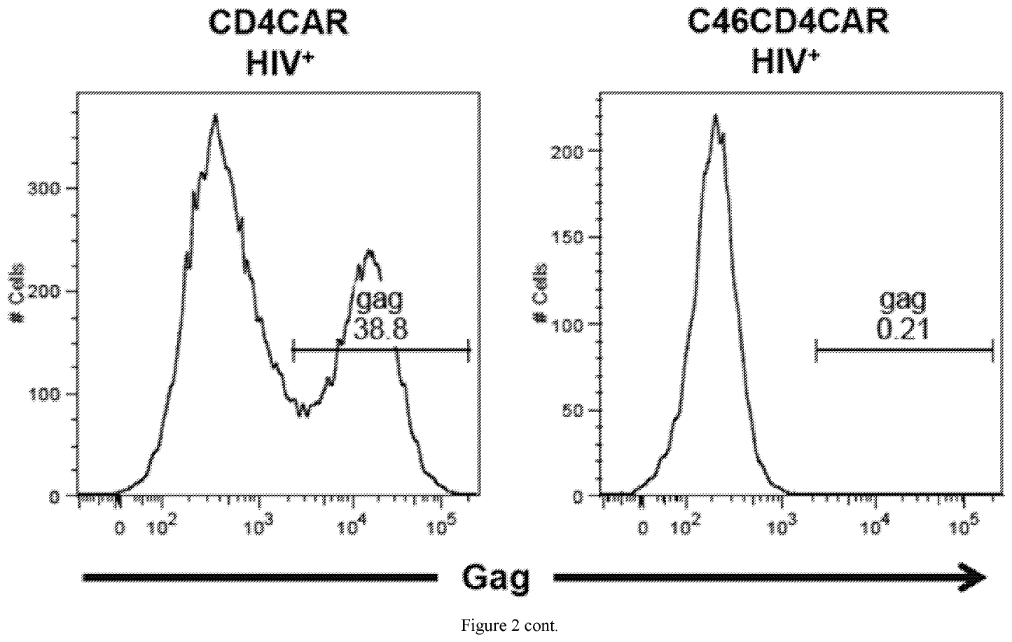

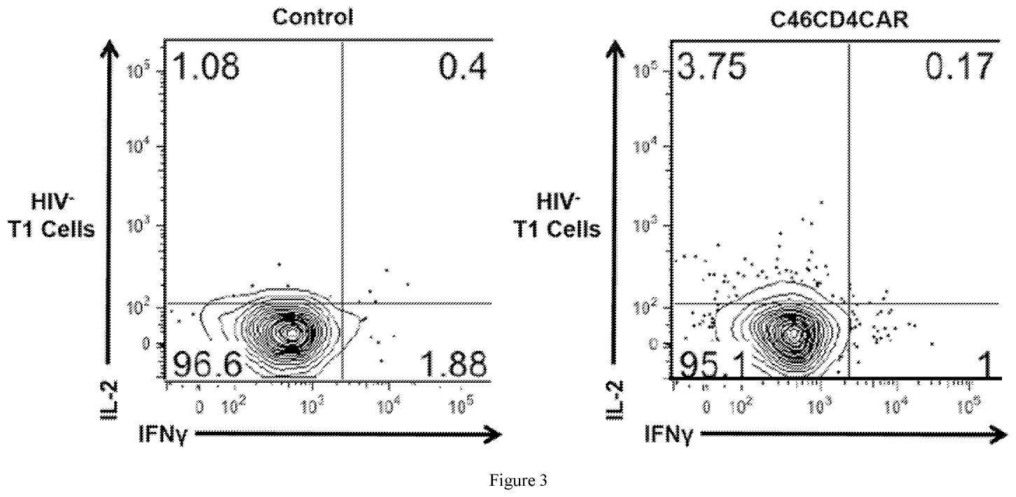

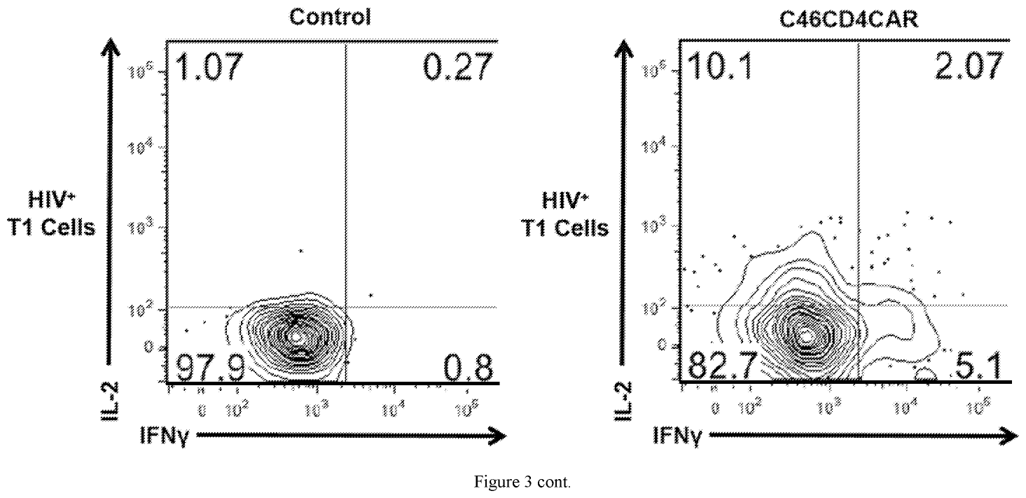

The CAR construct exemplified herein is an FG11 lentivirus vector that co-expresses an inhibitor of immunodeficiency virus fusion, i.e., the enfuvirtide-related peptide C46, and the human CD4 extracellular and transmembrane domain linked to the human CD3ζ signaling domain (C46CD4CAR). The expression of C46 is driven by the ubiquitin promoter and the expression of CD4CAR is driven by the EF-lalpha promoter. The control vector that was used contains C46 and a truncated form of CD4CAR that lacks the signaling domain of CD3ζ (C46CD4CARΔzeta). FIG. 1 schematically shows C46CD4CAR and C46CD4CARΔzeta. As shown in FIG. 2, expression of CD4CAR (a CAR without C46) resulted in increased HIV infection of Jurkat T cells (35.8% HIV⁺, as compared to 12% for unmodified cells). Expression of C46CD4CAR, however, blocked HIV infection (0.21% HIV⁺ cells), thereby indicating that C46 protected gene modified cells from HIV infection.

Therefore, in some embodiments, the present invention provides CAR constructs that co-express an inhibitor of immunodeficiency virus fusion such as C46, and engineered cells expressing a CAR construct that co-expresses an inhibitor of immunodeficiency virus fusion such as C46. In some embodiments, the CAR construct is C46CD4CAR. In some embodiments, the engineered cell is a HSPC-based CAR cell that co-expresses an inhibitor of immunodeficiency virus fusion such as C46. In some embodiments, the engineered cell is a HSPC-based C46CAR cell. In some embodiments, a CAR construct according to the present invention may include as an alternative to, or in addition to, C46, one or more co-stimulatory molecules, such as 41BB and/or CD28 (34).

Functional T-Cells Expressing C46CD4CAR

To determine whether a T-cell expressing C46CD4CAR can functionally respond to antigen, pigtail macaque T cells were transduced with C46CD4CAR or with C46CD4CARΔzeta and then stimulated with either uninfected or HIV-infected cells that express the HIV envelope. Cells transduced with C46CD4CAR produced IL-2 and IFNγ in response to stimulation, thereby indicating that T-cells expressing C46CD4CAR functionally respond to HIV-infected cells (FIG. 3). In contrast, control cells expressing C46CD4CARΔzeta did not respond to HIV-infected cells.

These data show that cells expressing C46CD4CAR are protected against HIV infection and respond functionally and specifically to HIV antigen.

Multilineage Engraftment of HSPC-Based C46CAR Cells

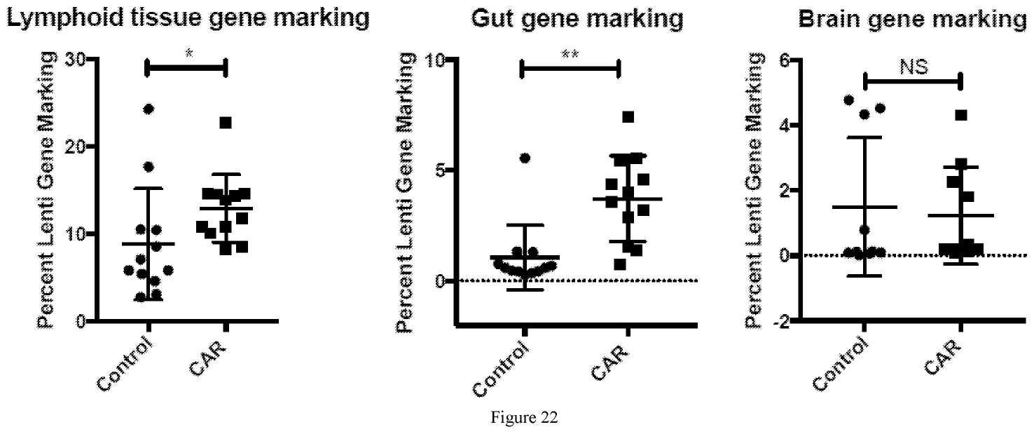

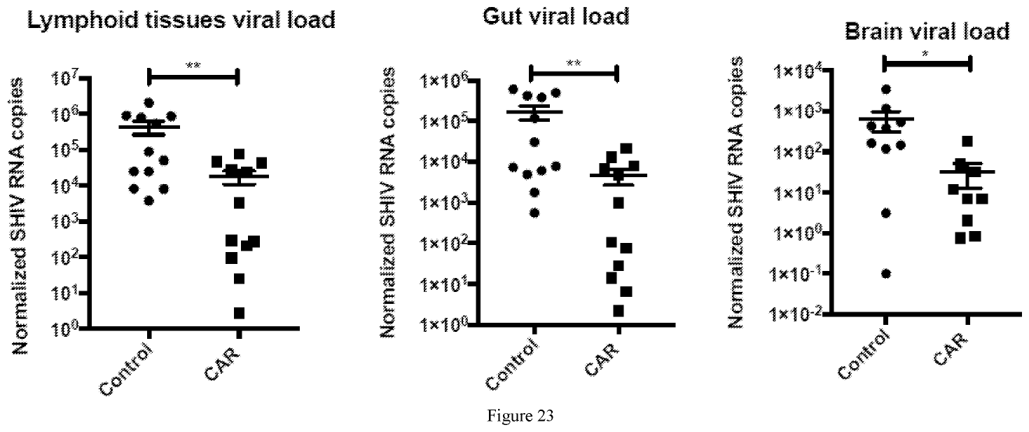

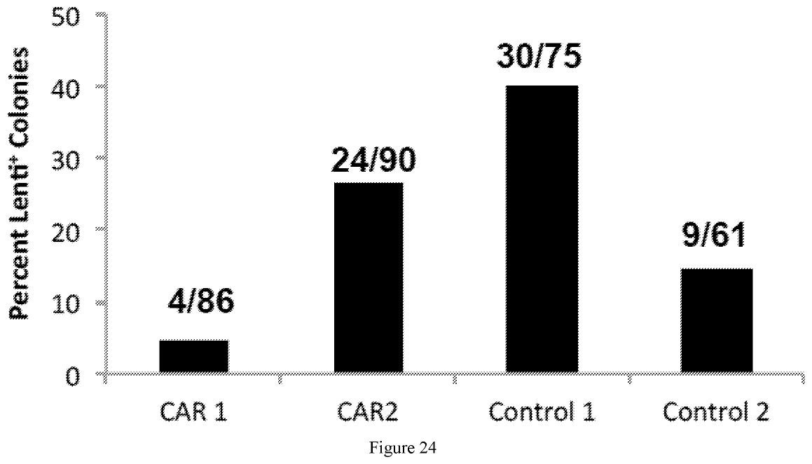

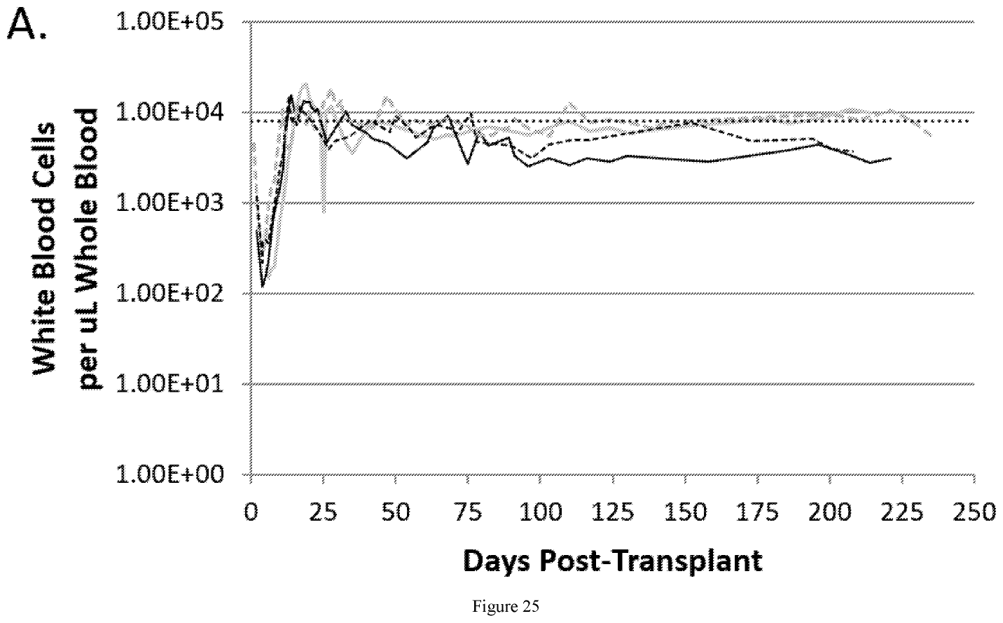

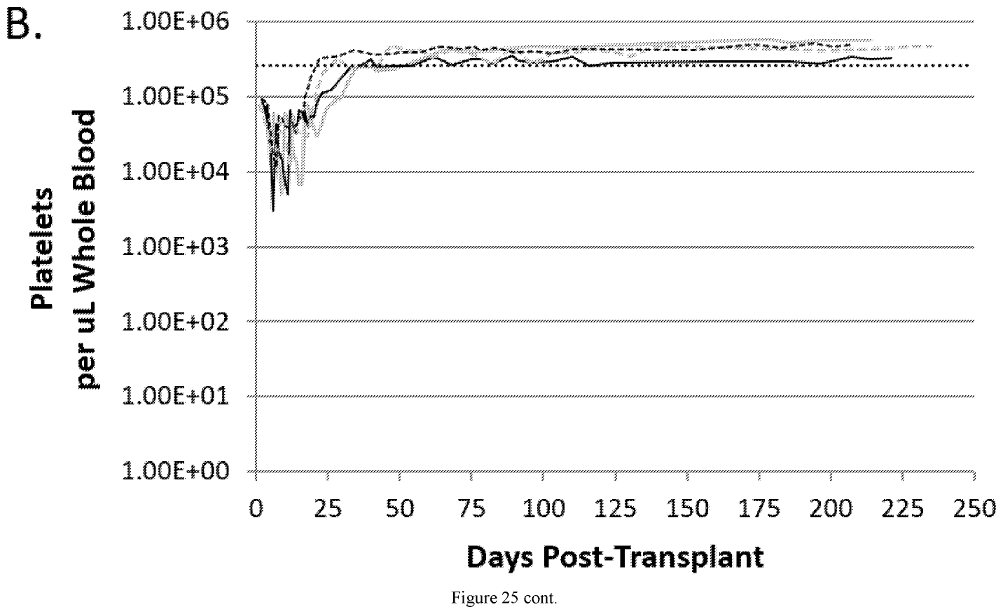

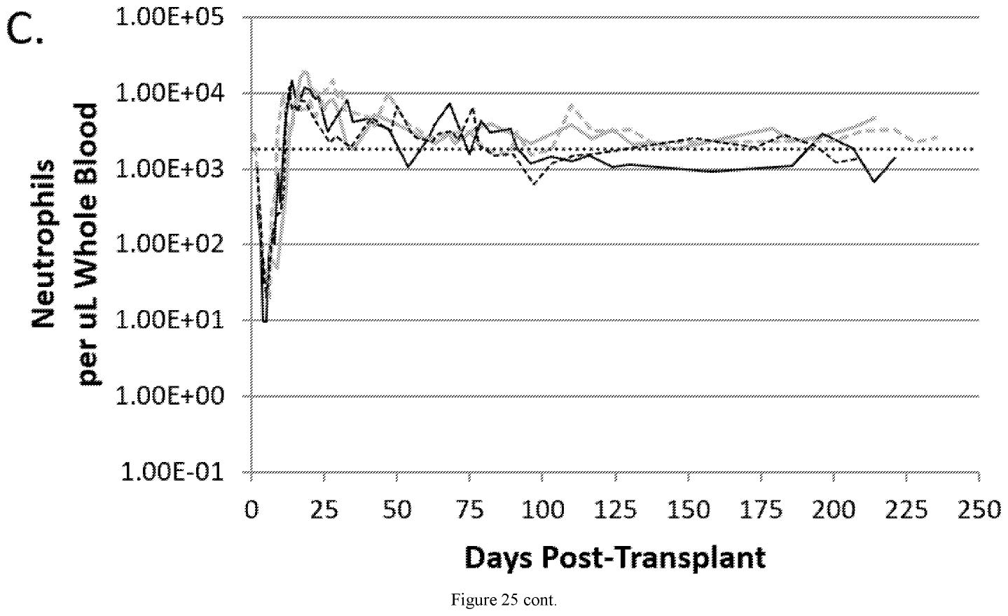

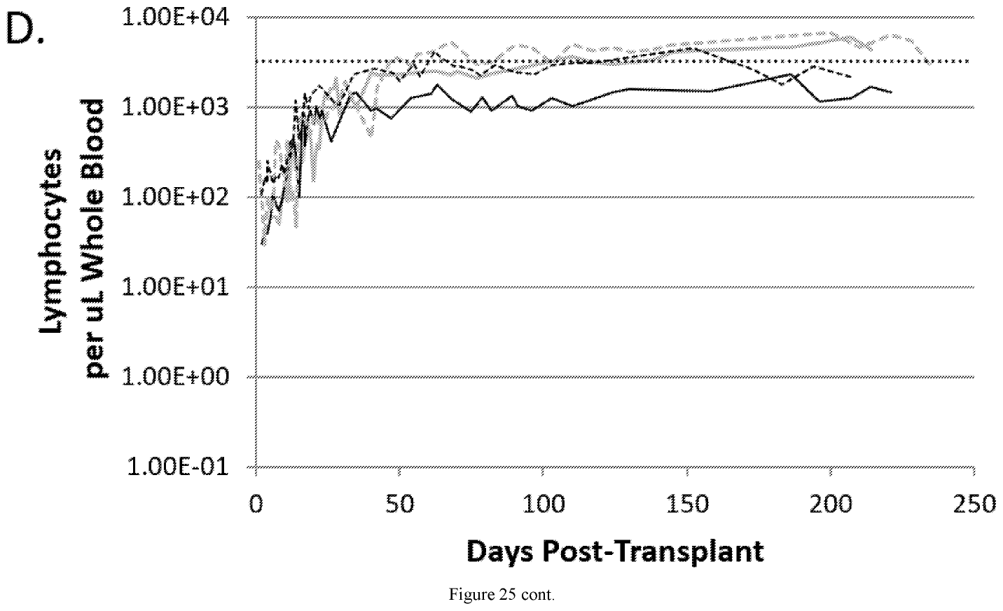

To examine the effects of the HSPC-based CAR cells in vivo, four male juvenile pigtail macaques were transplanted with autologous HSPCs that were transduced with C46CD4CAR (“CAR 1” and “CAR 2”) or C46CD4CARΔzeta (“Control 1” and “Control 2”). As used herein, a “control subject” refers to one that has been treated with an HSPC transduced with the control vector, i.e., C46CD4CARΔzeta, and a “C46 subject” refers to one that has been treated with HSPC-based C46CAR cells. As shown in FIG. 24, percent lentivirus marking from each subject's HSPC infusion product ranged from 4.65% to 40% in colony forming assays. After HSPC transplant, recovery kinetics of total white blood cells, platelets, neutrophils, and lymphocytes in both control subjects and C46 subjects were normal (FIG. 25).

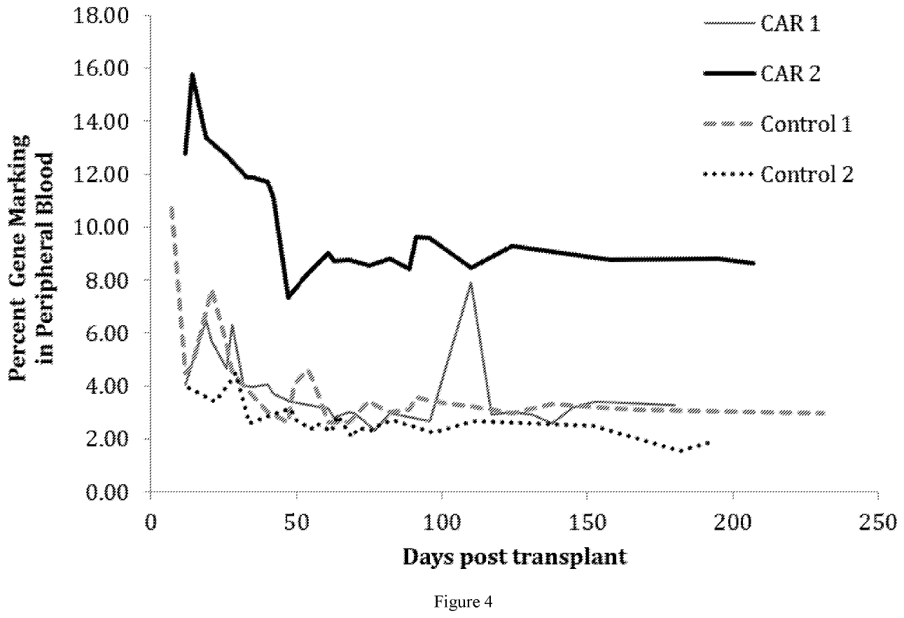

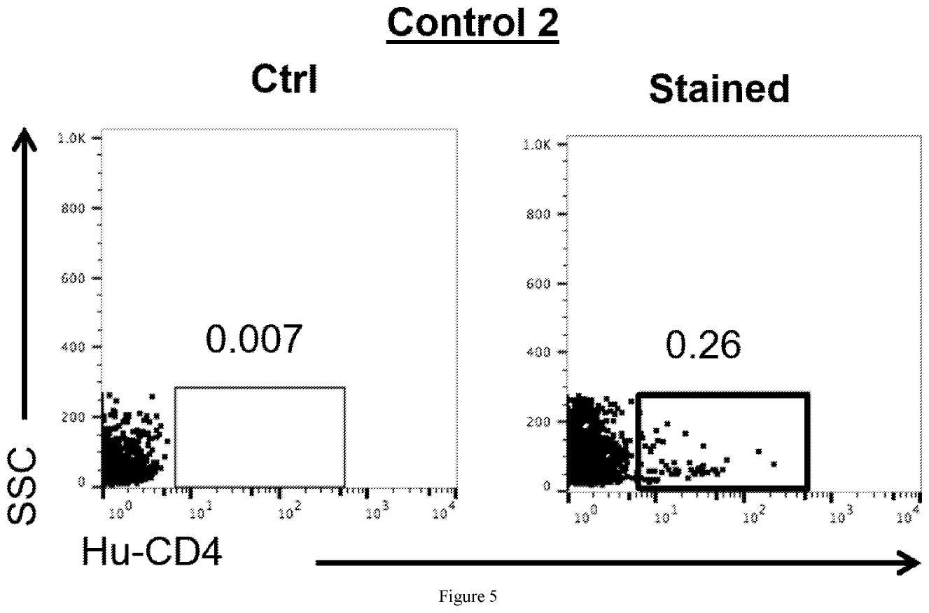

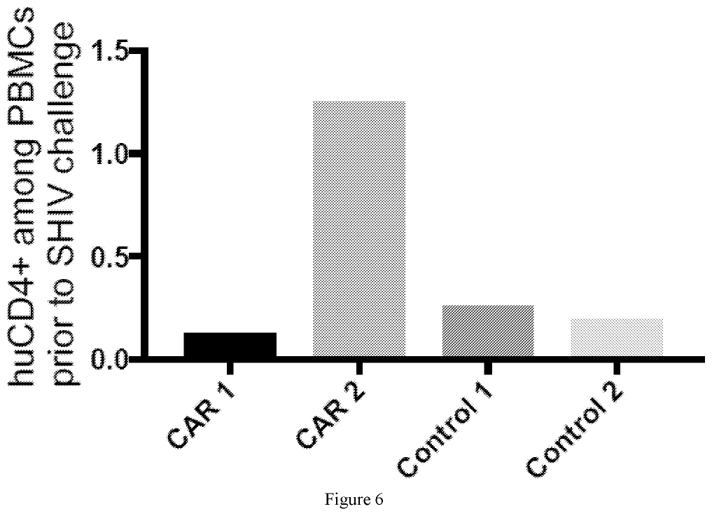

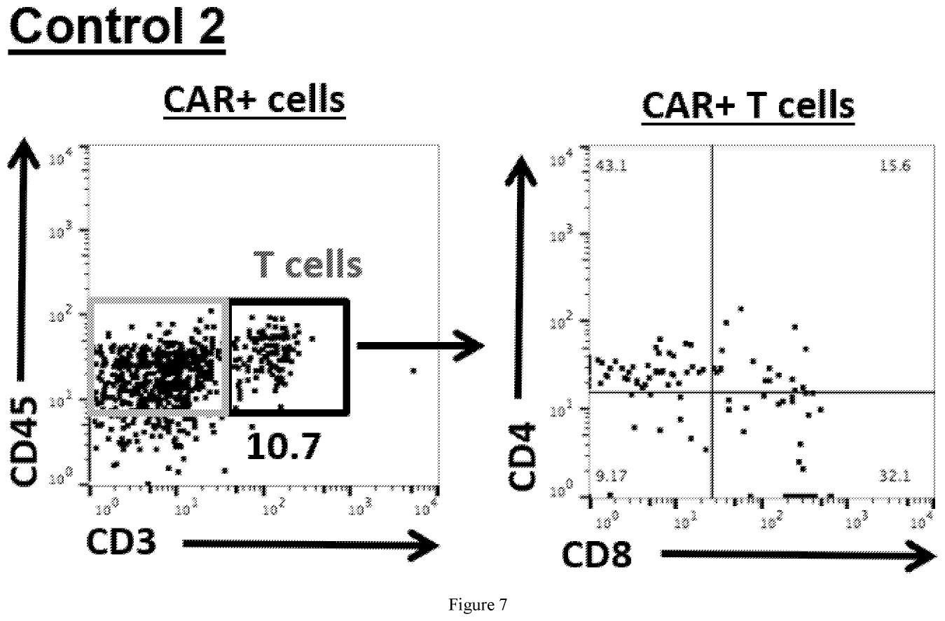

Stable gene marking of peripheral blood mononuclear cells (PBMCs) from all subjects was detected prior to challenge with SHIV (FIG. 4). In addition, cells expressing C46CD4CAR or C46CD4CARΔzeta in peripheral blood were detected using an anti-human CD4 antibody clone (13B8.2) that detects human, but not pigtail macaque CD4 (FIG. 5). Because this antibody will only label CARs expressing human CD4 in subjects, the CAR′ cells are “huCD4⁺”. It was found that 0.1% to 1.25% of CD45⁺ peripheral blood leukocytes from control subjects or C46 subjects were huCD4⁺ (FIG. 6). Importantly, huCD4⁺ cells from both control subjects and C46 subjects differentiated into multiple hematopoietic lineages, including T cells (CD45⁺CD3⁺), natural killer (NK) cells (CD3⁻CD2⁺NKG2A⁺), B cells (CD45⁺CD3⁻CD20⁺), and monocytes and macrophages (CD3⁻CD20⁻CD14⁺) (FIG. 7).

These results show that transplantation with HSPC-based CAR cells, including HSPC-based C46CAR cells, is safe and well tolerated, and results in stable, multilineage engraftment with typical kinetics of hematopoietic recovery. Therefore, in some embodiments, the present invention provides a HSPC-based CAR cell that is T-cell that expresses a CAR, which is referred to as a “HSPC-based CAR T-cell”. In some embodiments, the HSPC-based CAR T-cell expresses C46CD4CAR. In some embodiments, the present invention provides a HSPC-based CAR cell that is an NK cell that expresses a CAR, which is referred to as a “HSPC-based CAR NK cell”. In some embodiments, the HSPC-based CAR NK cell expresses a CAR that is designed for enhancing NK cell activity against HIV. In some embodiments, the present invention provides a HSPC-based CAR cell that is a B cell that expresses a CAR, which is referred to as a “HSPC-based CAR B cell”.

Multilineage Engraftment of HSPC-Based C46CAR Cells in Tissues

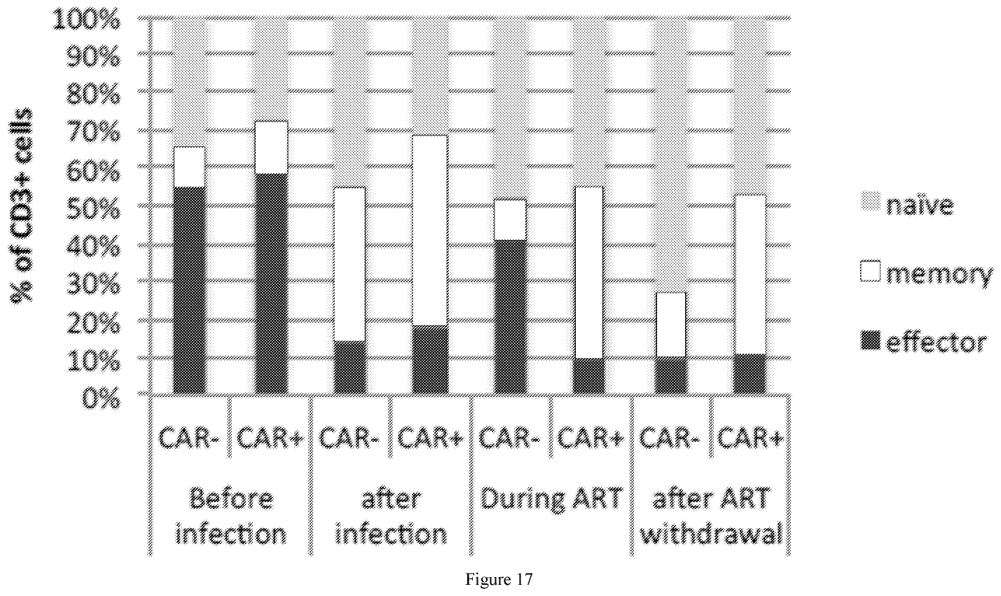

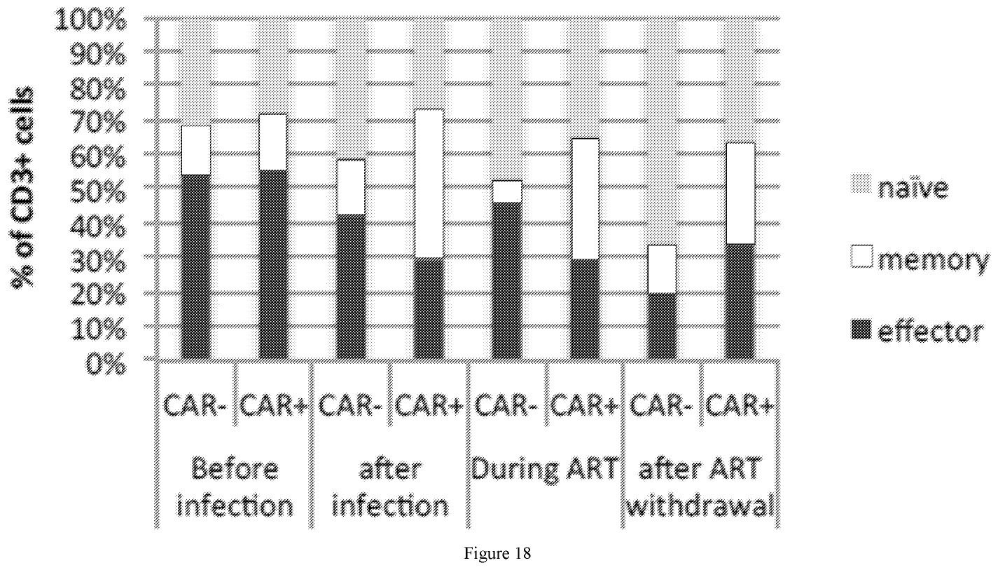

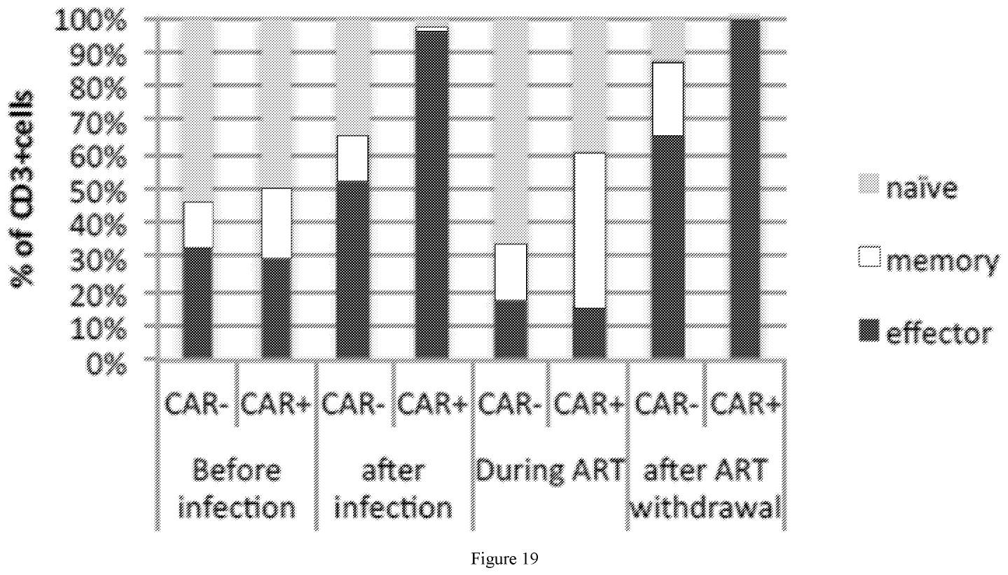

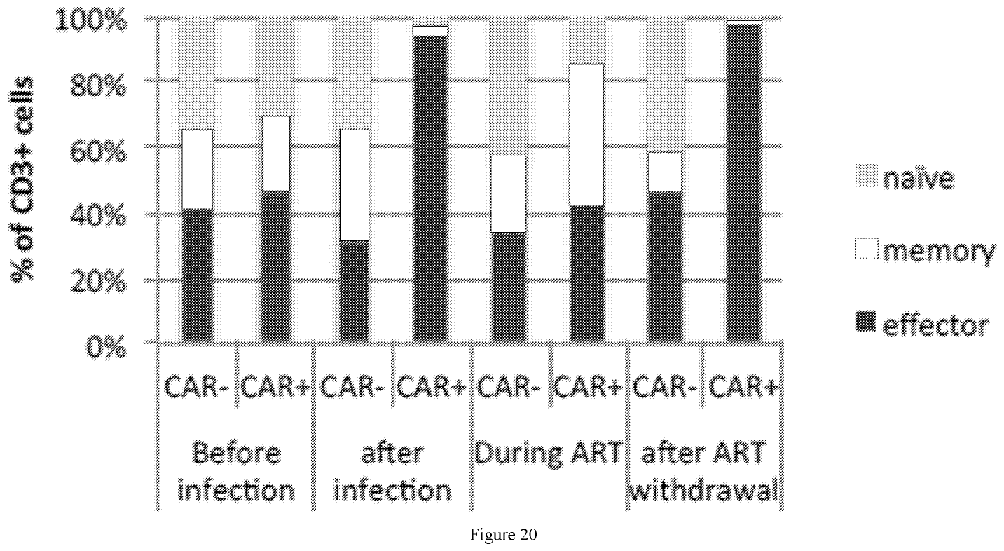

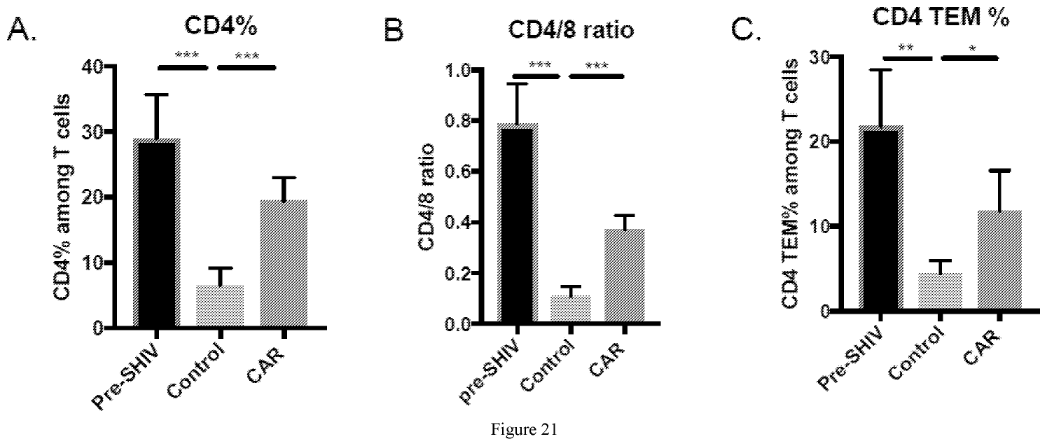

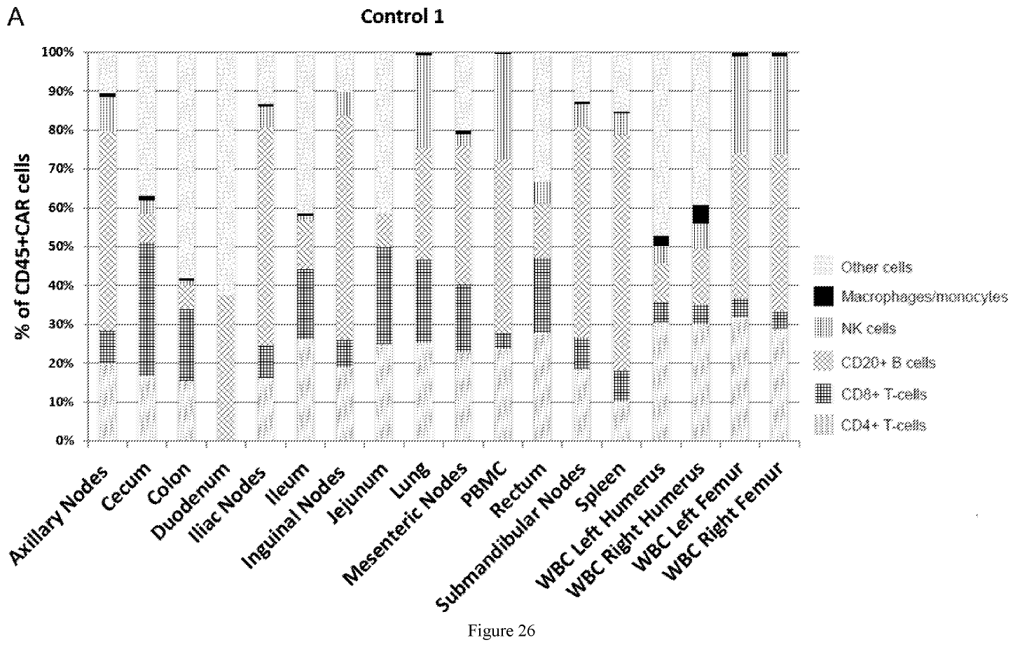

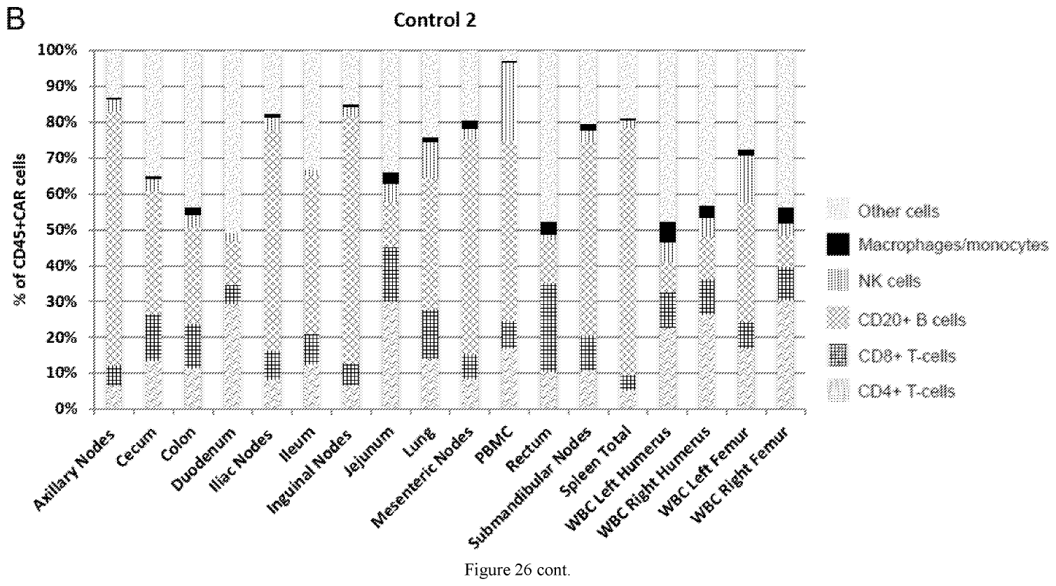

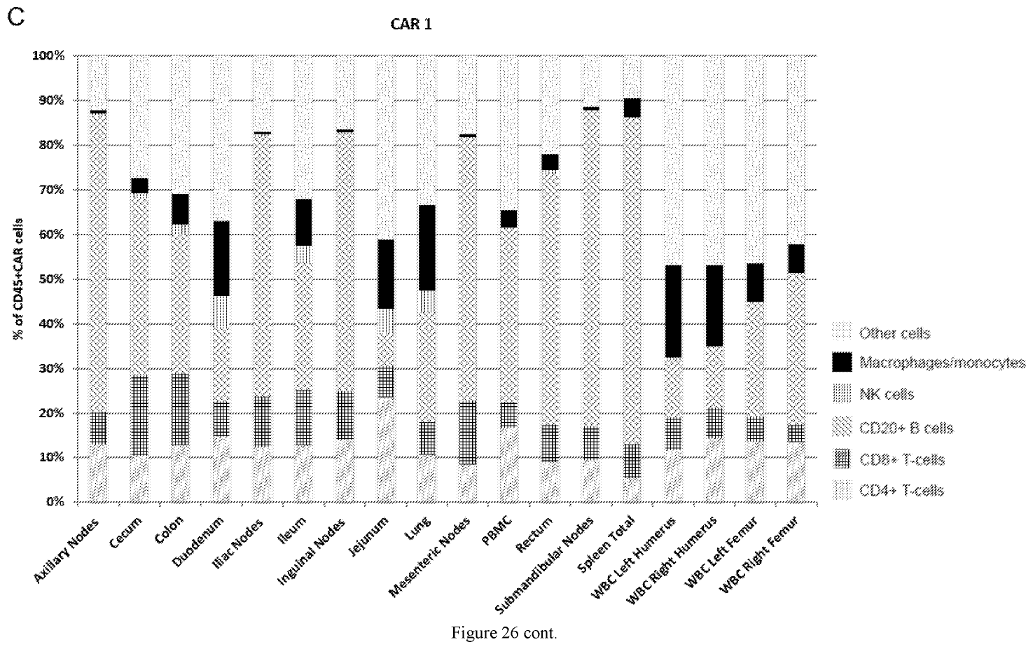

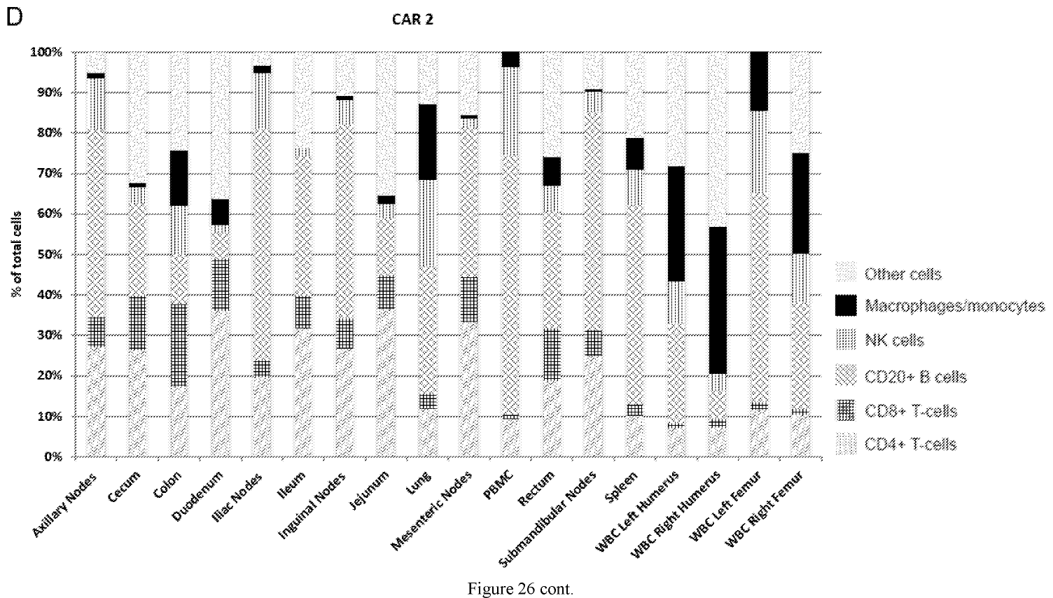

The trafficking of HSPC-based CAR cells to multiple tissue sites, including those that have been characterized as viral reservoirs, was examined. Cells expressing C46CD4CAR and C46CD4CARΔzeta were found in multiple lymphoid tissues, including various lymph nodes, gut, and bone marrow (FIG. 26). As with huCD4⁺PBMCs, tissue-associated cells expressing CARs were multilineage. There were no observable differences in cell composition between C46CD4CAR and C46CD4CARΔzeta expressing cells (FIG. 26). To examine the ability of HSPC-based C46CAR cells to protect against SHIV-dependent depletion of CD4⁺ cells in the gut, biopsies were taken from the GI tract (colon or duodenum/jejunum) before SHIV infection and after withdrawal of cART, and analyzed by flow cytometry. Control subjects displayed a profound loss of CD4⁺ cells, both in terms of CD4⁺CD3⁺ T cell percentage (FIG. 21, Panel A) and CD4/8 ratio (FIG. 21, Panel B).

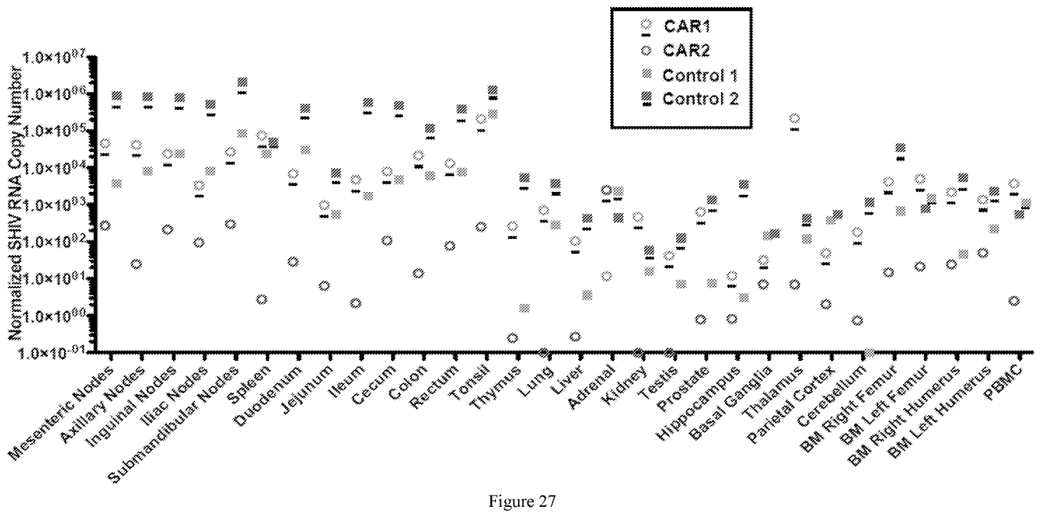

Strikingly, CD4⁺ T-cell percentages and CD4/CD8 ratios were significantly higher in C46 subjects following SHIV infection, suppression of cART, and withdrawal of cART, suggesting that functional T cells expressing C46CD4CAR contributed to protection of immune homeostasis in this compartment. Furthermore, CD4⁺ effector memory T cells (CD3⁺CD4⁺CCR7⁻CD45RA⁻), which are major target cells of HIV infection, were also protected in the gut of C46 subjects (FIG. 21, Panel C). At necropsy, gene marking in lymphoid tissues (including spleen, mesenteric lymph nodes, axillary lymph nodes, inguinal lymph nodes, and submandibular lymph nodes) and gut (including duodenum, jejunum, ileum, cecum, colon, and rectum) was significantly higher in C46 subjects relative to control subjects (FIG. 22). Interestingly, SHIV RNA measurements in these tissues showed that C46 subjects had significantly lower viral loads (FIG. 23). Although significant differences in gene marking in the brain (including hippocampus, basal ganglia, thalamus, parietal cortex, and cerebellum) between C46 subjects and control subjects was not observed, SHIV RNA measurements were also lower in this compartment in C46 subjects, relative to control subjects (FIG. 22 and FIG. 23). In particular, the CAR 2 subject had dramatically lower SHIV mRNA (4-5 logs) across all lymphoid tissues as compared to control subjects (FIG. 27).

Collectively, these results demonstrate that tissue cells expressing C46CD4CAR are capable of long term, multilineage engraftment, and are protected against viral replication, which is consistent with the observations in peripheral blood. Therefore, in some embodiments, the present invention provides one or more tissue cells that express a CAR, such as C46CD4CAR.

Viral Antigen Expansion of HSPC-Based C46CAR Cells In Vivo

Additionally, HSPC-based C46CAR cells expanded in response to SHIV infection in an antigen-driven fashion, and differentiated into effector cells in a CD3 domain-dependent manner. Intriguingly, the HSPC-based CAR cells contracted during cART and lower levels of antigen expression, and then rapidly expanded after withdrawal of cART in a manner mimicking a memory response. As a result, C46 subjects had decreased viremia during post-cART viral rebound, as compared to control subjects.

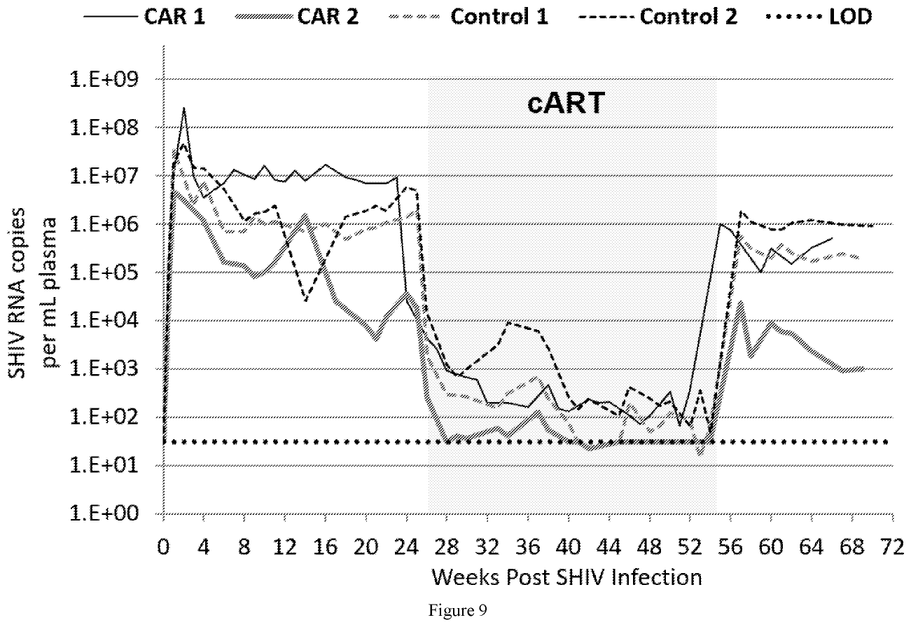

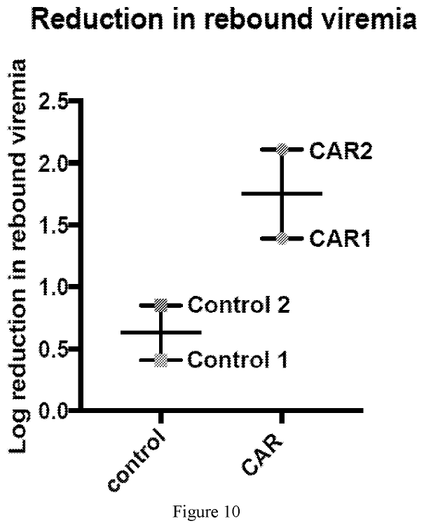

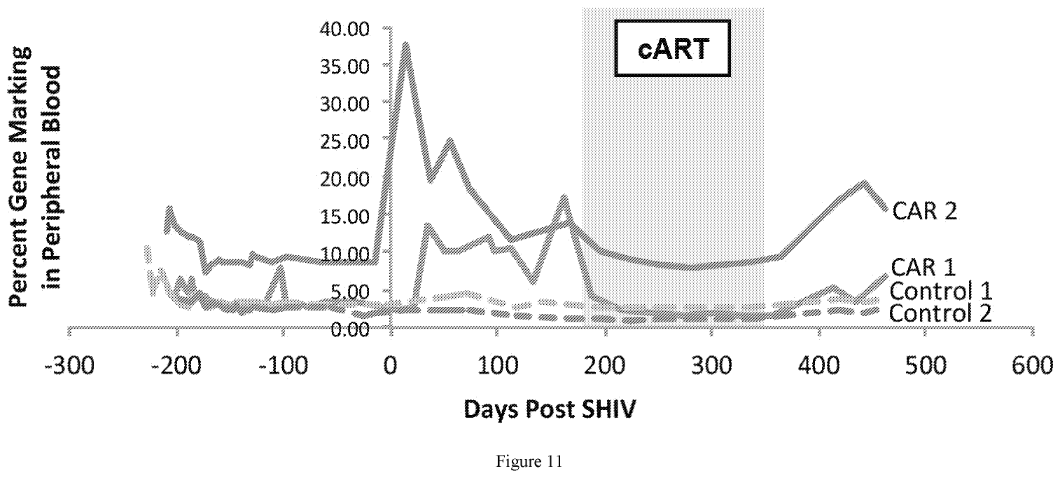

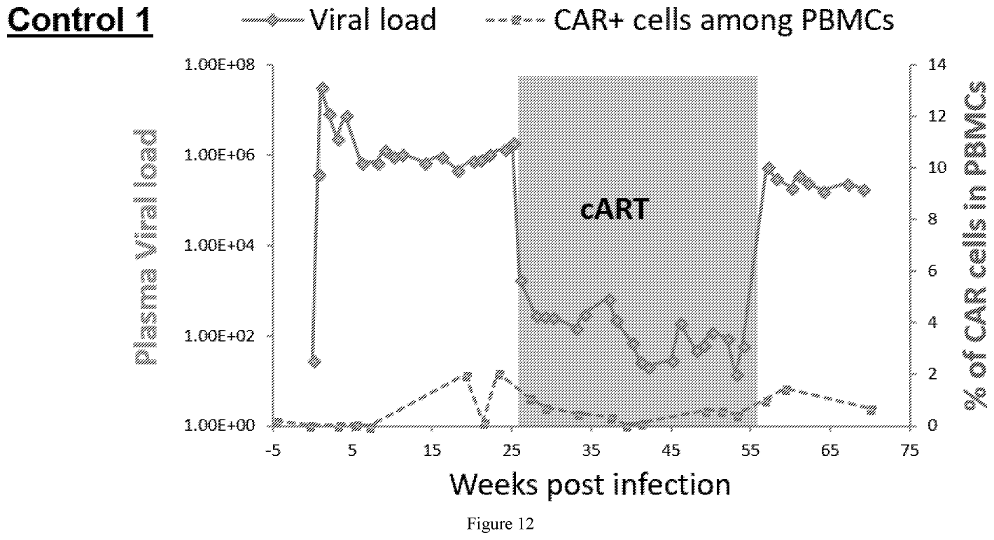

Specifically, antigen-dependent responses in C46 subjects and control subjects were measured by monitoring CAR gene marking as a function of SHIV plasma viremia. Lentivirus-marked cells were readily detectable by Taqman-based assays in C46 subjects and control subjects over the course of the study period of about two years (FIG. 11). Interestingly, C46 subjects, but not controls, showed increased gene marking in the periphery at multiple time points. These were coincident with increases in SHIV viremia, notably during primary infection and viral rebound after cART withdrawal (FIG. 9). To investigate further, flow cytometry was used to stain for huCD4⁺ PBMCs at multiple time points following SHIV infection. Consistent with Taqman-based gene marking data, huCD4⁺ cells from C46 subjects, but not control subjects, expanded upon SHIV infection and exhibited post-cART withdrawal viral rebound (FIG. 12 to FIG. 15). This confirms that cells expressing C46CD4CAR requires intact CD3ζ signaling to expand in response to viral antigen.

Furthermore, an increase of C46CD4CAR⁺ cells was observed during acute and chronic SHIV infection (FIG. 14 and FIG. 15), reminiscent of a primary immune response to infection. After the cessation of cART, the percentage of CD4CAR⁺ cells again increased rapidly, mimicking a memory response. The CAR 2 subject, which had higher gene marking prior to SHIV infection (FIG. 4), contained as many as 10% and 12.6% huCD4⁺ PBMCs during primary untreated infection and after cART withdrawal, respectively. During viral rebound, the 4-10 fold higher levels of CAR marking in C46 subjects relative to control subjects was consistent with the 1.5-2 log decrease in rebound viremia relative to primary infection in these subjects (FIG. 10).

These data suggest that HSPC-based C46CAR cells engraft long term and are capable of antigen-specific expansion months or years after transplantation. Therefore, in some embodiments, HSPC-based CAR cells, including HSPC-based C46CAR cells, are expanded in a subject by administering to the subject an effective amount of gp120 and/or one or more epitopes thereof. In some embodiments, the amount of gp120 and/or one or more epitopes thereof administered to the subject is an immunogenic amount. In some embodiments, HSPC-based CAR cells, including HSPC-based C46CAR cells, are expanded in a subject by administering to the subject an effective amount of an HIV vaccine, e.g., AIDSVAX, Modified Vaccinia Ankara B (MVA-B), ALVAC, or the like, that acts by binding human CD4. In some embodiments, where the viral load is relatively low in a subject, e.g., levels that are reduced from antiretroviral therapy, in order to maintain or increase the amount of HSPC-based CAR cells in the subject, an effective amount of gp120 and/or one or more epitopes thereof is administered to the subject. In some embodiments, the amount of gp120 and/or one or more epitopes thereof administered to the subject is an immunogenic amount. In some embodiments, where the viral load is relatively low in a subject, e.g., levels that are reduced from antiretroviral therapy, in order to maintain or increase the amount of HSPC-based CAR cells in the subject, an effective amount of an HIV vaccine, e.g., AIDSVAX, Modified Vaccinia Ankara B (MVA-B), ALVAC, or the like, that acts by binding human CD4 is administered to the subject.

Reduced SHIV Rebound in C46 Subjects

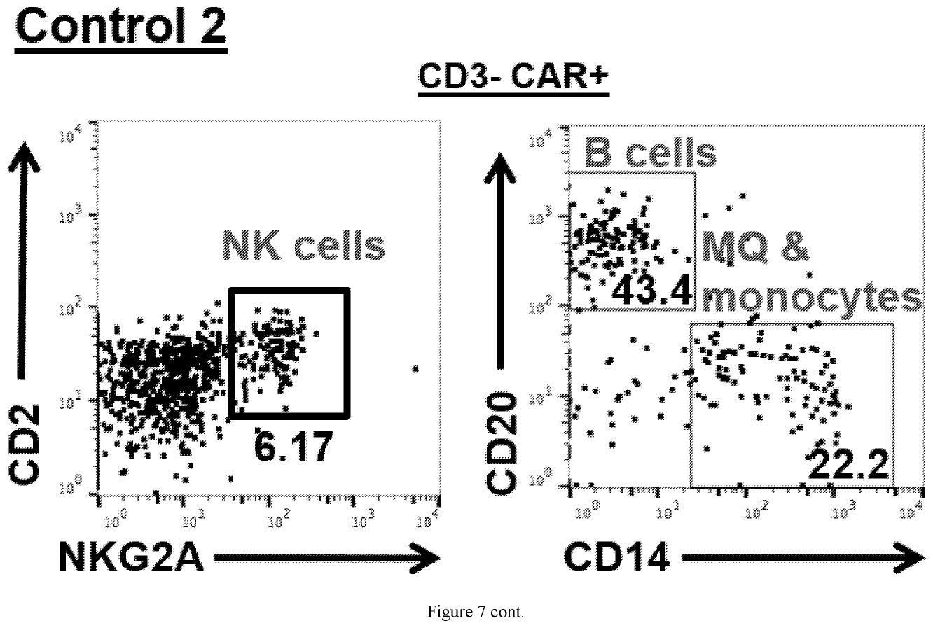

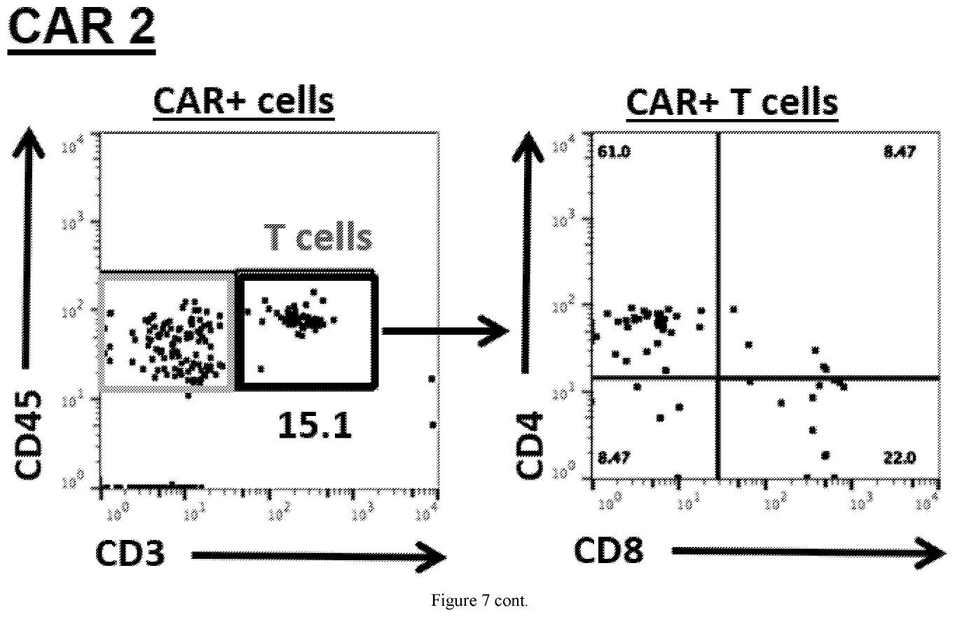

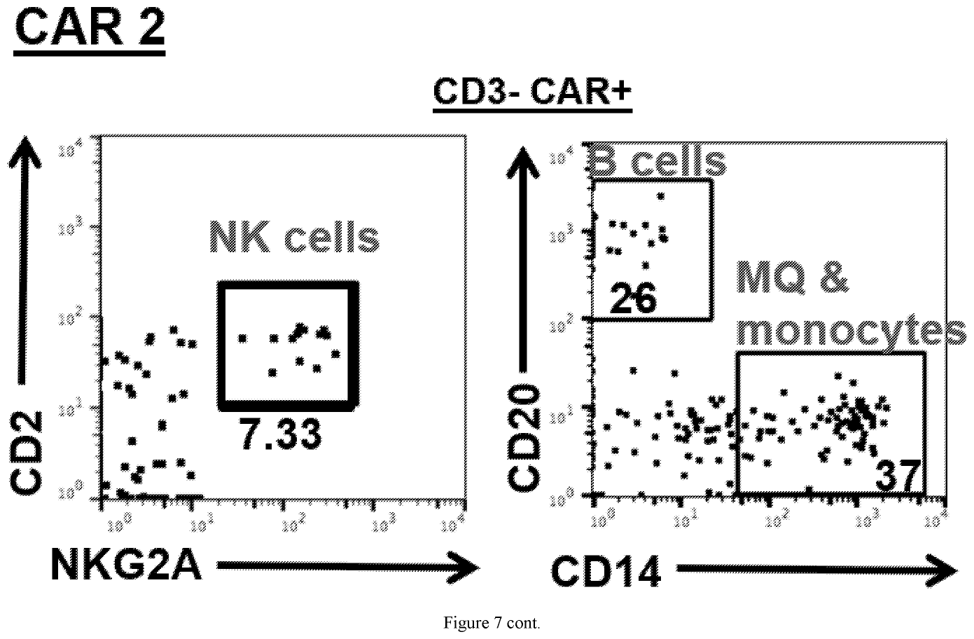

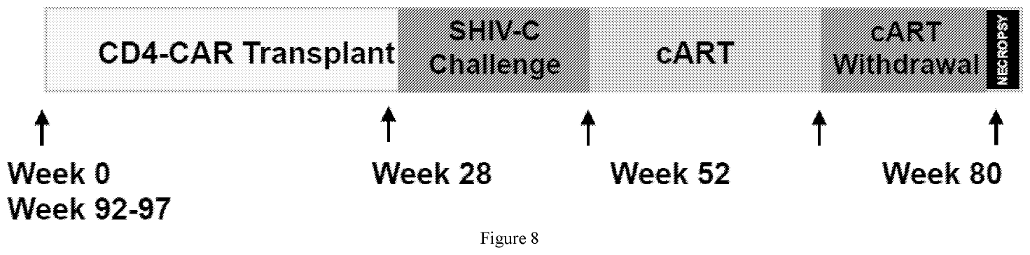

To study the effect of transplantation with HSPC-based C46CAR cells on SHIV replication, subjects were infected with SHIV-C for about 24 weeks followed by 28 weeks of cART and subsequent cART withdrawal. At least 12 weeks after cART cessation, subjects were then sacrificed for necropsy (FIG. 8). Both C46 subjects and control subjects had slightly higher plasma viral loads prior to cART, and did not achieve full virus suppression following cART (FIG. 9). This was likely due to residual immune suppression from the transplant procedure. The CAR 1 subject had approximately 1 log higher viral load as compared to control subjects during acute and chronic SHIV infection, while the CAR 2 subject, in which more than 1% of PBMCs were HSPC-based C46CAR cells prior to SHIV infection, had lower peak viremia during acute infection and showed progressively decreasing viral loads prior to cART (FIG. 9). Interestingly, after cART withdrawal, both C46 subjects had lower average rebound viremia (1.4-2.11 log lower than primary setpoint) as compared to the control subjects (0.4-0.8 log lower than primary setpoint) (FIG. 10).

These findings indicate that HSPC-based C46CAR cells are capable of establishing virus-specific immune memory and responding to recrudescent viremia.

SHIV-Dependent Effector Differentiation of CD4CAR Cells

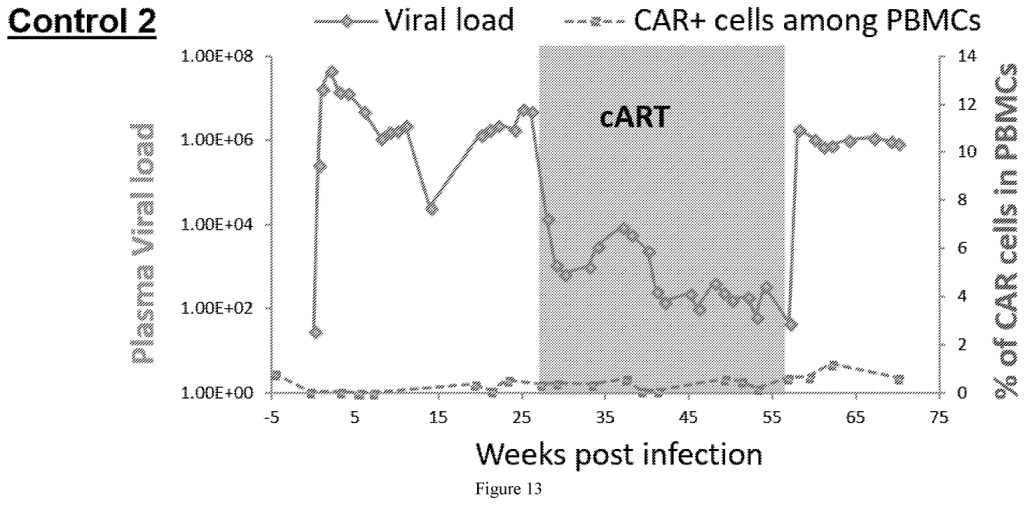

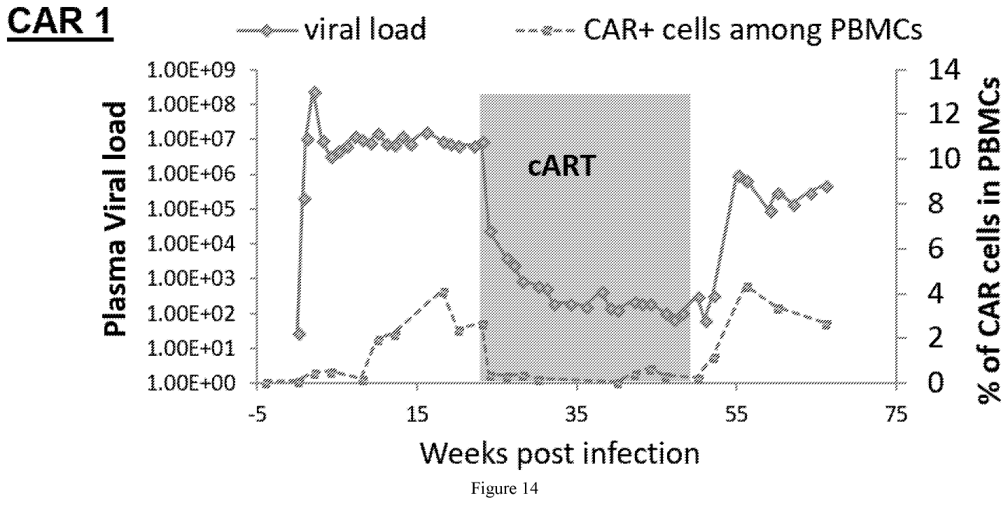

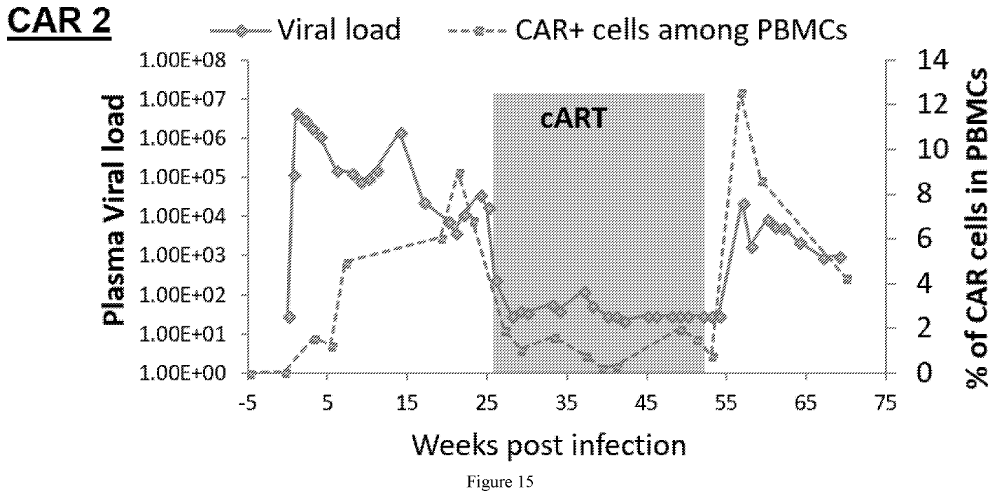

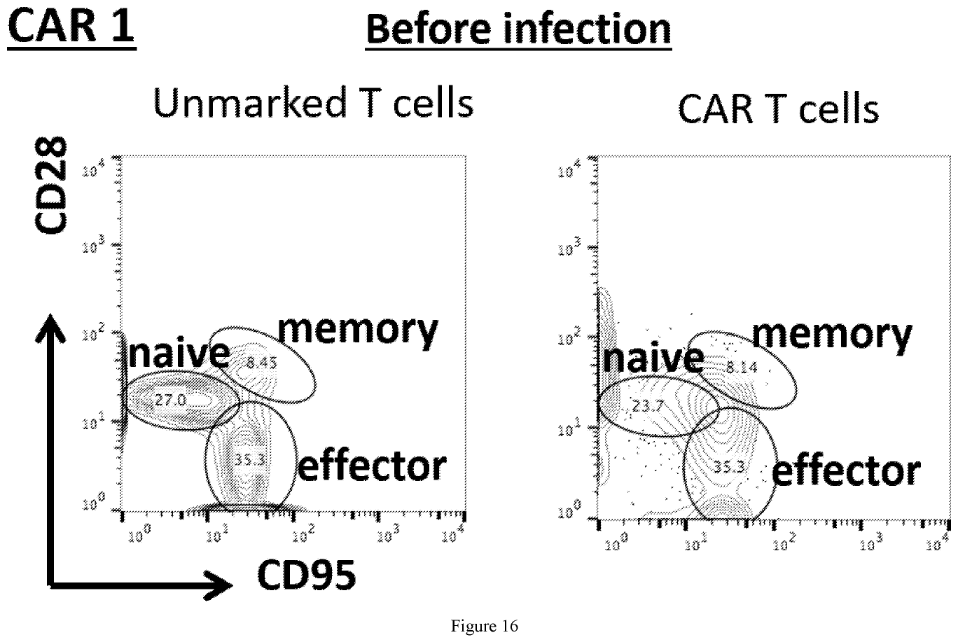

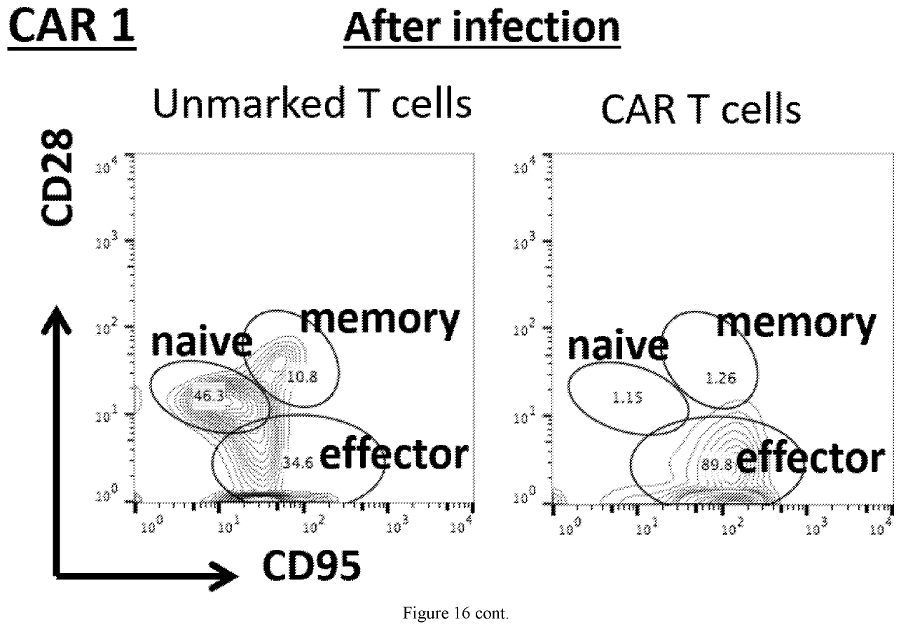

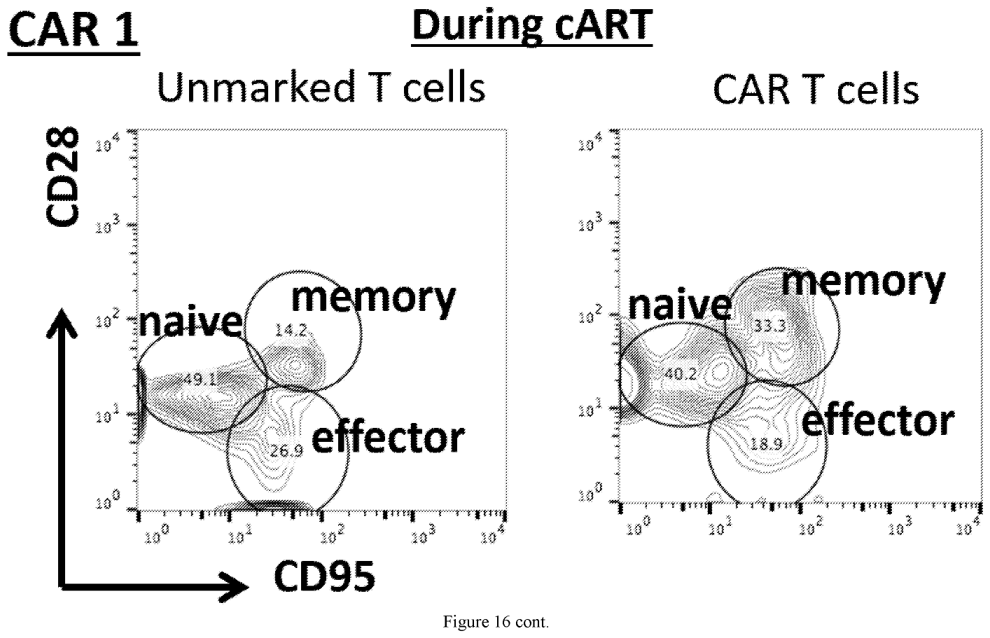

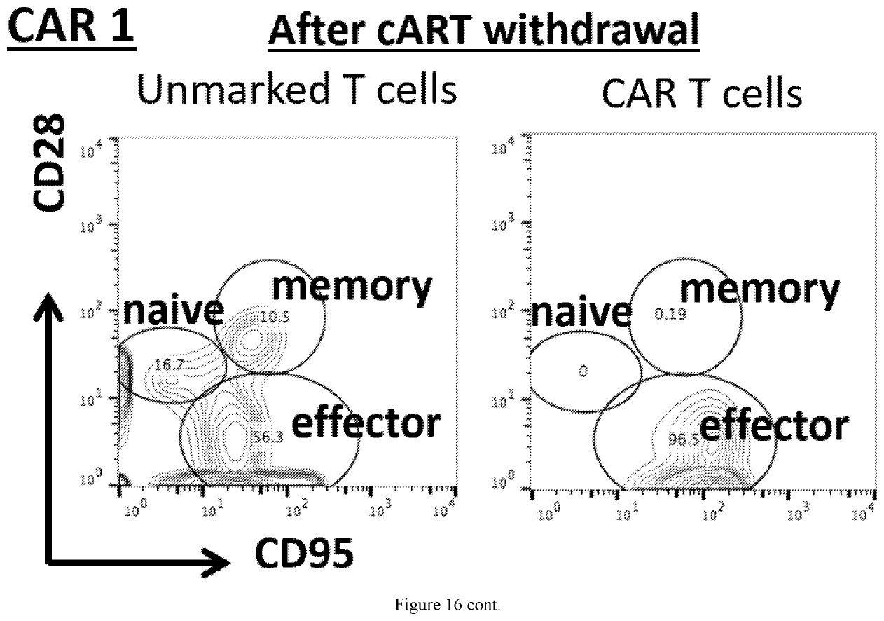

To examine how HSPC-based C46CAR cells respond to SHIV replication in vivo, the naïve, effector, and memory phenotypes of T cells were measured longitudinally in HSPC transplant subjects following SHIV infection. Prior to SHIV infection, huCD4⁺ (CAR⁺) and unmarked T cells (CAR⁻) shared similar percentages of naïve (CD28⁺CD95⁻), effector (CD28⁻CD95⁺) and memory (CD28⁺CD95⁺) subsets (FIG. 16). Strikingly, huCD4⁺ cells became predominantly effector T cells after SHIV infection, consistent with a response to SHIV antigen. During cART-dependent viral suppression, when the percentage of huCD4⁺ T cells contracted, most displayed a naïve or memory phenotype. After cART withdrawal, huCD4⁺ T cells again displayed a predominant effector phenotype. Antigen-dependent increases in the percentage of effector cells were observed in C46 subjects, but not in control subjects (FIG. 17 to FIG. 20). For both C46 subjects, robust expansion of HSPC-based C46CAR cells after SHIV infection and cART withdrawal, which was dependent on CD3ζ signaling, was observed. The immediate, memory like response of the HSPC-based C46CAR cells after cART withdrawal from both C46 subjects likely contributed to improved control of SHIV viremia and CD4 protection in the gut as compared to control subjects.

These findings demonstrate that HSPC-based C46CAR cells generate a long-lived, functional response to viral antigen in subjects.

Multifaceted HSPC-Based CAR Cell Treatments

Because HSPCs transduced with CAR constructs are found to differentiate into HSPC-based CAR T-cells and HSPC-based CAR NK cells, in some embodiments, a mixture of HSPCs transduced with different CAR constructs, e.g., a CAR construct that expresses C46CD4CAR, and a CAR construct that expresses a CAR that enhances NK cell activity against HIV, e.g., increased NK cell activation in the presence of HIV, is transplanted in a subject.

In some embodiments, HSPC-based CAR cells according to the present invention are used in combination with one or more latency reversing agents and/or additive immunotherapies to eradicate viral infection and provide an effective immune surveillance for HIV in subjects. In some embodiments, before administration of a latency reversing agent, the HSPC-based CAR cells in the subject are expanded by administering to the subject an effective amount of an agent that binds human CD4, such as gp120 and/or one or more epitopes thereof. In some embodiments, the subject is administered an immunogenic amount of gp120 and/or one or more epitopes thereof. In some embodiments, the agent is an HIV vaccine, e.g., AIDSVAX, Modified Vaccinia Ankara B (MVA-B), ALVAC, or the like, that acts by binding human CD4. Examples of latency reversing agents include byrostatin, histone deacetylase inhibitors (e.g., vorinostat, panobinostat, romidepsin, etc.), toll-like receptor 7 (TLR7) agonists (e.g., GS-9620), and the like.

In some embodiments, before exposure to and/or infection by HIV or SHIV, one or more HSPC-based CAR cells are transplanted into a subject, and then the subject is administered an effective amount of an agent that binds human CD4, such as gp120 and/or one or more epitopes thereof. In some embodiments, the subject is administered an immunogenic amount of gp120 and/or one or more epitopes thereof. In some embodiments, the agent is an HIV vaccine, e.g., AIDSVAX, Modified Vaccinia Ankara B (MVA-B), ALVAC, or the like, that acts by binding human CD4. In some embodiments, if the subject to be treated is on antiretroviral therapy, the antiretroviral therapy is withdrawn prior to administration of the effective amount of the agent.

In some embodiments, the subject is administered one or more antiretroviral therapeutics such as Abacavir (ZIAGEN), Atazanavir (REYATAZ), ATRIPLA (efavirenz, FTC, tenofovir), Darunavir (PREZISTA), DESCOVY (tenofovir alafenamide, emtricitabine), Dolutegravir (TIVICAY), Efavirenz (SUSTIVA), Elvitegravir (VITEKTA), Emtricitabine (FTC, EMTRIVA), Etravirine (INTELENCE), EVIPLERA (rilpivirine, emtricitabine, and tenofovir), EVOTAZ (atazanavir and cobicistat), Fosamprenavir (TELZIR), GENVOYA (elvitegravir, cobicistat, emtricitabine, tenofovir alafenamide (TAF)), KIVEXA (abacavir/3TC), KIVEXA (lamivudine, abacavir), Lamivudine (3TC, EPIVIR), KALETRA (lopinavir, ritonavir), Maraviroc (CELSENTRI), Nevirapine (VIRAMUNE), Odefsey (rilpivirine, emtricitabine, tenofovir alafenamide (TAF)), Raltegravir (ISENTRESS), Rezolsta (darunavir, cobicistat), Rilpivirine (EDURANT), Ritonavir (NORVIR), STRIBILD (elvitegravir, emtricitabine, tenofovir disoproxil, cobicistat), Tenofovir (VIREAD), Triumeq (dolutegravir, abacavir, lamivudine), Truvada (tenofovir, FTC), Zidovudine (AZT, RETROVIR), and the like.

In some embodiments, HSPC-based CAR cells according to the present invention are transplanted in subjects to prophylactically treat the subjects against being infected with HIV. For example, in some embodiments a subject who is not infected with HIV is treated with HSPCs transduced with a CAR construct that encodes, e.g., C46CD4CAR. The transplanted HSPCs are allowed to differentiate and develop into mature naive T cells that express the CAR and then the HSPC-based CAR T-cells are expanded into effector and memory T cells by administering to the subject an effective amount of an agent that binds human CD4, such as gp120 and/or one or more epitopes thereof. In some embodiments, the subject is administered an immunogenic amount of gp120 and/or one or more epitopes thereof. In some embodiments, the agent is an HIV vaccine, e.g., AIDSVAX, Modified Vaccinia Ankara B (MVA-B), ALVAC, or the like, that acts by binding human CD4.

Compositions

In some embodiments, compositions according to the present invention comprise one or more HSPC-based CAR cells, such as HSPC-based C46CAR cells. In some embodiments, compositions according to the present invention are pharmaceutical compositions. In some embodiments, the pharmaceutical compositions comprise a therapeutically effective amount of one or more HSPC-based CAR cells, such as HSPC-based C46CAR cells. As used herein, a “pharmaceutical composition” refers to a composition suitable for pharmaceutical use in a subject. A pharmaceutical composition generally comprises an effective amount of an active agent and a pharmaceutically acceptable carrier, e.g., a buffer, adjuvant, and the like. As used herein, a “pharmaceutically acceptable carrier” refers to solvents, dispersion media, coatings, antibacterial and antifungal agents, isotonic and absorption delaying agents, and the like, that are compatible with the active ingredient and comply with the applicable standards and regulations, e.g., the pharmacopeial standards set forth in the United States Pharmacopeia and the National Formulary (USP-NF) book, for pharmaceutical administration. Thus, for example, unsterile water is excluded as a pharmaceutically acceptable carrier for, at least, intravenous administration. Pharmaceutically acceptable carriers include those in the art. See, e.g., REMINGTON: THE SCIENCE AND PRACTICE OF PHARMACY. 20th ed. (2000) Lippincott Williams & Wilkins. Baltimore, Md.

As used herein, an “effective amount” refers to a dosage or amount sufficient to produce a desired result. The desired result may comprise an objective or subjective response in, for example, a treatment group as compared to a control group in, for example, an in vitro assay. In some embodiments, the effective amount is a “therapeutically effective amount”. As used herein, a “therapeutically effective amount” refers to an amount sufficient to effect a beneficial or desired therapeutic (including preventative) result in a subject, such as a reduction of HIV infected cells and/or suppression of HIV viral replication, as compared to a control or a baseline measurement before treatment. Therapeutically effective amount and immunogenic amounts may be administered as a single dose or as a series of several doses. As used herein, an “immunogenic amount” is an amount that is sufficient to elicit an immune response in a subject and depends on a variety of factors such as the immunogenicity of the given antigen, the manner of administration, the general state of health of the subject, and the like. The skilled artisan will appreciate that certain factors may influence the dosage required to effectively treat or immunize a subject, including the degree of symptoms, previous treatments, the general health and age of the subject, and the like. Nevertheless, effective amounts and therapeutically effective amounts may be readily determined using methods in the art.

Pharmaceutical compositions of the present invention may be formulated for the intended route of delivery, including intravenous, intramuscular, intraperitoneal, subcutaneous, intraocular, intrathecal, intraarticular, intrasynovial, cisternal, intrahepatic, intralesional injection, intracranial injection, infusion, and/or inhaled routes of administration using methods in the art. Pharmaceutical compositions according to the present invention may include one or more of the following: pH buffered solutions, adjuvants (e.g., preservatives, wetting agents, emulsifying agents, and dispersing agents), liposomal formulations, nanoparticles, dispersions, suspensions or emulsions as well as sterile powders for reconstitution into sterile injectable solutions or dispersions. The compositions and formulations of the present invention may be optimized for increased stability and efficacy using methods in the art.

Dosages and Regimen

Pharmaceutical compositions of the present invention may be provided in dosage unit forms. As used herein, “dosage unit form” refers to physically discrete units suited as unitary dosages for the subject to be treated; each unit containing a predetermined quantity of an active ingredient calculated to produce the desired therapeutic effect in association with the required pharmaceutically acceptable carrier. The specification for the dosage unit forms of the invention are dictated by the unique characteristics of the active ingredient and the particular therapeutic effect to be achieved, and the limitations inherent in the art of compounding such an active ingredient for the treatment of individuals.

Toxicity and therapeutic efficacy of the compositions according to the present invention can be determined by standard pharmaceutical procedures in cell cultures or experimental animals. For example, one may determine the lethal dose, LC₅₀ (the dose expressed as concentration x exposure time that is lethal to 50% of the population) or the LD₅₀ (the dose lethal to 50% of the population), and the ED₅₀ (the dose therapeutically effective in 50% of the population) by methods in the art. The dose ratio between toxic and therapeutic effects is the therapeutic index and it can be expressed as the ratio LD₅₀/ED₅₀. Compositions that exhibit large therapeutic indices are preferred. While compositions that exhibit toxic side effects may be used, care should be taken to use a delivery system that targets such compositions to the site of affected tissue in order to reduce side effects.

The data obtained from the cell culture assays and animal studies can be used in formulating a range of dosages for various combinations of one or more compositions of the present invention for use in humans. The dosages are preferably within a range of circulating concentrations that include the ED₅₀ with little or no toxicity. The dosage may vary within this range depending upon the dosage form employed and the route of administration utilized. A therapeutically effective dose can be estimated from cell culture assays. A dose may be formulated in animal models to achieve a circulating plasma concentration range that includes the IC₅₀ (i.e., the concentration of the test composition which achieves a half-maximal inhibition of symptoms) as determined in cell culture. Such information can be used to more accurately determine useful doses in humans. Levels in plasma may be measured using methods in the art.

Additionally, a suitable dosage for a given subject can be determined by an attending physician or other qualified medical personnel, based on various clinical factors. As is well known in the medical arts, dosages for any one subject depend upon many factors, including the subject's size, body surface area, age, the particular compound to be administered, sex of the subject, time, and route of administration, general health, and other drugs being administered concurrently. Those of skilled in the art will readily appreciate that dose levels can vary as a function of the specific composition, e.g., the specific HSPC-based CAR cell, the severity of the symptoms and the susceptibility of the subject to side effects. Nevertheless, preferred dosages may be readily determined by those of skill in the art.

Materials and Methods

Autologous Transplantation of Pigtail Macaques

Four juvenile male pigtail macaques were transplanted with autologous, lentivirus modified HSPCs as previously described (14). The pigtail macaques (M. nemestrina), carry a TRIMS genotype that is permissive to lentivirus-mediated gene therapy approaches (15). The subjects were mobilized with granulocyte-colony stimulating factor (G-CSF) and stem cell factor (SCF) for 4 days prior to collection of large volume bone marrow aspirates and bead-based positive selection of CD34⁺ cells. Over a 48-hour ex vivo culture period, cells were transduced twice with the lentiviruses schematically shown in FIG. 1 at a multiplicity of infection (MOI) of 5 (CAR 1) or 10 (CAR 2, Control 1, Control 2). During HSPC transduction ex vivo, subjects received a myeloablative conditioning regimen consisting of a fractionated dose of 1020 cGy total body irradiation. Following conditioning, the HSPC product was infused back into the autologous subject. A small aliquot of the infused cell product was plated in Colony Gel Medium (Reach Bio, Seattle, Wash.) and analyzed as previously described (14, 36). Individual colonies and total genomic DNA (gDNA) isolated at the indicated post-transplant time points were measured by gel-based and Taqman-based PCR methods, respectively, as previously described (14, 17).

SHIV Infection, cART, and Peripheral Blood/Tissue Analyses

Subjects were allowed to recover for approximately 200 days prior to infection with SHIV-C(SHIV-1157ipd3N4), which was administered to subjects via the intravenous challenge route as previously described (11). Combination antiretroviral therapy (cART) consisted of 20 mg/kg Tenofovir and 40 mg/kg Emtricitabine (FTC) dosed once per day subcutaneously, and 150 mg Raltegravir dosed twice per day orally with food. Plasma viral loads, peripheral T-cell counts, longitudinal tissue surgeries, and necropsy tissue collections were conducted as previously described (12, 14). Taqman-based peripheral blood measurements were performed from gDNA isolated from total leukocytes. Total RNA and gDNA from tissue samples were isolated using a Precellys 24 homogenizer and CK28-R hard tissue homogenizing beads (Bertin Corp.) as previously described (12). PCR-based assays for SHIV were designed not to detect HIV-based lentiviral vectors (data not shown).

SHIVs, including SHIV-C, are simian immunodeficiency viruses (SIVs) that contain the envelope of human immunodeficiency virus (HIV). As such, CARs, expressing human CD4, such as C46CD4CAR, will bind the envelope of HIVs as well as SHIV as evidenced by the in vitro and in vivo experiments herein. SHIV-C was used in the in vivo experiments herein to examine the characteristics, activity, and function of HSPC-based CAR cells that express human CD4 because HIV does not infect non-human primates. Nevertheless, because SHIV-C contains HIV envelope, the experiments and results herein are applicable to human subjects and HIV.

Flow Cytometry

To detect huCD4⁺ cells, PBMCs and tissue necropsy samples were stained with the following antibodies: anti-human specific CD4 antibody (for detection and analysis of cells expressing human CD4; Beckman Coulter, clone 13B8.2), anti-NHP CD45 (BD Biosciences, clone D058-1283), anti-CD4 (eBiosciences, clone OKT4), anti-CD8 (eBiosciences, clone SK1), anti-CD20 (eBiosciences, clone 2H7), anti-NK2Ga (Beckman Coulter, clone A60797), anti-CD14 (Beckman Coulter, clone IM2707U), anti-CD95 (BD Biosciences, clone DX2), anti-CD28 (BD Biosciences, clone D28.2), and anti-CD3 (BD Biosciences, clone SP34-2). Fluorophore conjugates included FITC, PE, PE-Cy5, PE-Cy7, alexa700, V500, efluor450, APC, and APC-efluor780.

Cytokine Assay

PBMCs were purified from healthy pigtail macaque blood and stimulated with bead bound anti-CD3 (BD Biosciences, clone SP34-2) and anti-CD28 (BD Biosciences, clone D282.2) for 3 days. Afterwards, cells were transduced with either C46CD4CAR or C46CD4CARΔzeta lentivirus. 2 days after transduction, cells were co-incubated with either T1 cells or HIV-infected T1 cells for 16 hours, followed by 6 hours of Golgi Plug treatment. Afterwards cells were first surface-stained with anti-CD3, anti-human CD4 (for detection of CD4CAR transduced cells) and then intracellular-stained with anti-IFNγ (eBiosciences, clone 4S B3), anti-IL-2 (BD Biosciences, clone MQ1-17H12) and analyzed by flow cytometry.

In Vitro HIV Infection

Jurkat cells were either untransduced or transduced with 2M01 CD4CAR (CAR construct without C46) or C46CD4CAR for 2 days and infected with HIV-1NL4.3 (100 ngp 24/10 cells) for 3 days. Afterwards, cells were intracellularly stained with anti-Gag (clone KC57) and analyzed by flow cytometry.

### REFERENCES

The following references are herein incorporated by reference in their entirety:
1. O. O. Yang, A. C. Tran, S. A. Kalams, R. P. Johnson, M. R. Roberts, B. D. Walker, Lysis of HIV-1-infected cells and inhibition of viral replication by universal receptor T cells, Proc Natl Acad Sci USA 94, 11478-11483 (1997).
2. S. G. S. Deeks, B. B. Wagner, P. A. P. Anton, R. T. R. Mitsuyasu, D. T. D. Scadden, C. C. Huang, C. C. Macken, D. D. D. Richman, C. C. Christopherson, C. H. C. June, R. R. Lazar, D. F. D. Broad, S. S. Jalali, K. M. K. Hege, A Phase II Randomized Study of HIV-Specific T-Cell Gene Therapy in Subjects with Undetectable Plasma Viremia on Combination Antiretroviral Therapy, Mol Ther 5, 10-10 (2002).
3. R. T. R. Mitsuyasu, P. A. P. Anton, S. G. S. Deeks, D. T. D. Scadden, E. E. Connick, M. T. M. Downs, A. A. Bakker, M. R. M. Roberts, C. H. C. June, S. S. Jalali, A. A. A. Lin, R. R. Pennathur-Das, K. M. K. Hege, Prolonged survival and tissue trafficking following adoptive transfer of CD4zeta gene-modified autologous CD4(+) and CD8(+) T cells in human immunodeficiency virus-infected subjects, Blood 96, 785-793 (2000).
4. A. Zhen, M. Kamata, V. Rezek, J. Rick, B. Levin, S. Kasparian, I. S. Chen, O. O. Yang, J. A. Zack, S. G. Kitchen, HIV-specific Immunity Derived From Chimeric Antigen Receptor-engineered Stem Cells, Mol Ther 23, 1358-1367 (2015).
5. L. Liu, B. Patel, M. H. Ghanem, V. Bundoc, Z. Zheng, R. A. Morgan, S. A. Rosenberg, B. Dey, E. A. Berger, Novel CD4-Based Bispecific Chimeric Antigen Receptor Designed for Enhanced Anti-HIV Potency and Absence of HIV Entry Receptor Activity, J. Virol. 89, 6685-6694 (2015).
6. A. Zhen, S. Kitchen, Stem-cell-based gene therapy for HIV infection, Viruses 6, 1-12 (2014).
7. S. G. S. Kitchen, B. R. B. Levin, G. G. Bristol, V. V. Rezek, S. S. Kim, C. C. Aguilera-Sandoval, A. A. Balamurugan, O. O. O. Yang, J. A. J. Zack, In vivo suppression of HIV by antigen specific T cells derived from engineered hematopoietic stem cells, PLoS Pathog 8, e1002649-e1002649 (2012).
8. E. Gschweng, S. De Oliveira, D. B. Kohn, Hematopoietic stem cells for cancer immunotherapy, Immunol Rev 257, 237-249 (2014).
9. O. Ho, K. Larsen, P. Polacino, Y. Li, D. Anderson, R. Song, R. M. Ruprecht, S.-L. Hu, Pathogenic infection of Macaca nemestrina with a CCR5-tropic subtype-C simian-human immunodeficiency virus, Retrovirology 6, 65-65 (2009).
10. D. L. Bolton, A. Pegu, K. Wang, K. McGinnis, M. Nason, K. Foulds, V. Letukas, S. D. Schmidt, X. Chen, J. P. Todd, J. D. Lifson, S. Rao, N. L. Michael, M. L. Robb, J. R. Mascola, R. A. Koup, G. Silvestri, Ed. Human Immunodeficiency Virus Type 1 Monoclonal Antibodies Suppress Acute Simian-Human Immunodeficiency Virus Viremia and Limit Seeding of Cell-Associated Viral Reservoirs, J. Virol. 90, 1321-1332 (2015).
11. C. W. Peterson, P. Younan, P. S. Polacino, N. J. Maurice, H. W. Miller, M. Prlic, K. R. Jerome, A. E. Woolfrey, S.-L. Hu, H.-P. Kiem, Robust suppression of env-SHIV viremia in Macaca nemestrina by 3-drug ART is independent of timing of initiation during chronic infection, J. Med. Primatol. 42, 237-246 (2013).
12. C. W. Peterson, C. Benne, P. Polacino, J. Kaur, C. E. McAllister, A. Filali-Mouhim, W. Obenza, T. A. Pecor, M.-L. Huang, A. Baldessari, R. D. Murnane, A. E. Woolfrey, K. R. Jerome, S.-L. Hu, N. R. Klatt, S. DeRosa, R. P. Sékaly, H.-P. Kiem, Loss of immune homeostasis dictates SHIV rebound after stem-cell transplantation, JCI Insight 2, e91230 (2017).
13. C. W. Peterson, J. Wang, K. K. Norman, Z. K. Norgaard, O. Humbert, C. K. Tse, J. J. Yan, R. G. Trimble, D. A. Shivak, E. J. Rebar, P. D. Gregory, M. C. Holmes, H.-P. Kiem, Long-term multilineage engraftment of autologous genome-edited hematopoietic stem cells in nonhuman primates, Blood 127, 2416-2426 (2016).
14. C. W. Peterson, K. G. Haworth, B. P. Burke, P. Polacino, K. K. Norman, J. E. Adair, S.-L. Hu, J. S. Bartlett, G. P. Symonds, H.-P. Kiem, Multilineage polyclonal engraftment of Cal-1 gene-modified cells and in vivo selection after SHIV infection in a nonhuman primate model of AIDS, Mol Ther Methods Clin Dev 3, 16007 (2016).
15. G. Brennan, Y. Kozyrev, T. Kodama, S.-L. Hu, Novel TRIMS isoforms expressed by Macaca nemestrina, J Virol. 81, 12210-12217 (2007).
16. P. M. Younan, P. Polacino, J. P. Kowalski, C. W. Peterson, N. J. Maurice, N. P. Williams, O. Ho, G. D. Trobridge, D. von Laer, M. Prlic, B. C. Beard, S. DeRosa, S.-L. Hu, H.-P. Kiem, Positive selection of mC46-expressing CD4+ T cells and maintenance of virus specific immunity in a primate AIDS model, Blood 122, 179-187 (2013).
17. B. C. Beard, G. D. Trobridge, C. Ironside, J. S. McCune, J. E. Adair, H.-P. Kiem, Efficient and stable MGMT-mediated selection of long-term repopulating stem cells in nonhuman primates, J Clin. Invest. 120, 2345-2354 (2010).
18. D. Mavilio, J. Benjamin, D. Kim, G. Lombardo, M. Daucher, A. Kinter, E. Nies-Kraske, E. Marcenaro, A. Moretta, A. S. Fauci, Identification of NKG2A and NKp80 as specific natural killer cell markers in rhesus and pigtailed monkeys, Blood 106, 1718-1725 (2005).
19. C. W. Peterson, K. G. Haworth, P. Polacino, M.-L. Huang, C. Sykes, W. M. Obenza, A. C. Repetto, A. Kashuba, R. Bumgarner, S. C. DeRosa, A. E. Woolfrey, K. R. Jerome, J. I. Mullins, S.-L. Hu, H.-P. Kiem, Lack of viral control and development of combination antiretroviral therapy escape mutations in macaques after bone marrow transplantation, AIDS 29, 1597-1606 (2015).
20. S. L. Lamers, R. Rose, E. Maidji, M. Agsalda-Garcia, D. J. Nolan, G. B. Fogel, M. Salemi, D. L. Garcia, P. Bracci, W. Yong, D. Commins, J. Said, N. Khanlou, C. H. Hinkin, M. V. Sueiras, G. Mathisen, S. Donovan, B. Shiramizu, C. A. Stoddart, M. S. McGrath, E. J. Singer, F. Kirchhoff, Ed. HIV DNA Is Frequently Present within Pathologic Tissues Evaluated at Autopsy from Combined Antiretroviral Therapy-Treated Patients with Undetectable Viral Loads, J. Virol. 90, 8968-8983 (2016).
21. H. Xu, X. Wang, N. Malam, P. P. Aye, X. Alvarez, A. A. Lackner, R. S. Veazey, G. Silvestri, Ed. Persistent Simian Immunodeficiency Virus Infection Drives Differentiation, Aberrant Accumulation, and Latent Infection of Germinal Center Follicular T Helper Cells, J. Virol. 90, 1578-1587 (2015).
22. Y. Fukazawa, R. Lum, A. A. Okoye, H. Park, K. Matsuda, J. Y. Bae, S. I. Hagen, R. Shoemaker, C. Deleage, C. Lucero, D. Morcock, T. Swanson, A. W. Legasse, M. K. Axthelm, J. Hesselgesser, R. Geleziunas, V. M. Hirsch, P. T. Edlefsen, M. Piatak, J. D. Estes, J. D. Lifson, L. J. Picker, B cell follicle sanctuary permits persistent productive simian immunodeficiency virus infection in elite controllers, Nat. Med. 21, 132-139 (2015).
23. G. Hütter, D. Nowak, M. Mossner, S. Ganepola, A. Müßig, K. Allers, T. Schneider, J. Hofmann, C. Kiicherer, O. Blau, I. W. Blau, W. K. Hofmann, E. Thiel, Long-term control of HIV by CCR5 Delta32/Delta32 stem-cell transplantation, N. Engl. J. Med. 360, 692-698 (2009).
24. T. J. Henrich, Z. Hu, J. Z. Li, G. Sciaranghella, M. P. Busch, S. M. Keating, S. Gallien, N. H. Lin, F. F. Giguel, L. Lavoie, V. T. Ho, P. Armand, R. J. Soiffer, M. Sagar, A. S. Lacasce, D. R. Kuritzkes, Long-term reduction in peripheral blood HIV type 1 reservoirs following reduced-intensity conditioning allogeneic stem cell transplantation, J INFECT DIS 207, 1694-1702 (2013).
25. P. Tebas, D. Stein, G. Binder-Scholl, R. Mukherjee, T. Brady, T. Rebello, L. Humeau, M. Kalos, E. Papasavvas, L. J. Montaner, D. Schullery, F. Shaheen, A. L. Brennan, Z. Zheng, J. Cotte, V. Slepushkin, E. Veloso, A. Mackley, W. T. Hwang, F. Aberra, J. Zhan, J. Boyer, R. G. Collman, F. D. Bushman, B. L. Levine, C. H. June, Antiviral effects of autologous CD4 T cells genetically modified with a conditionally replicating lentiviral vector expressing long antisense to HIV, Blood 121, 1524-1533 (2013).
26. B. P. Burke, B. R. Levin, J. Zhang, A. Sahakyan, J. Boyer, M. V. Carroll, J. C. Colon, N. Keech, V. Rezek, G. Bristol, E. Eggers, R. Cortado, M. P. Boyd, H. Impey, S. Shimizu, E. L. Lowe, G.-E. E. Ringpis, S. G. Kim, D. N. Vatakis, L. R. Breton, J. S. Bartlett, I. S. Y. Chen, S. G. Kitchen, D. S. An, G. P. Symonds, Engineering Cellular Resistance to HIV-1 Infection InVivo Using a Dual Therapeutic Lentiviral Vector, Mol Ther Nucleic Acids 4, e236 (2015).
27. L. Li, L. Krymskaya, J. Wang, J. Henley, A. Rao, L.-F. Cao, C.-A. Tran, M. Torres-Coronado, A. Gardner, N. Gonzalez, K. Kim, P.-Q. Liu, U. Hofer, E. Lopez, P. D. Gregory, Q. Liu, M. C. Holmes, P. M. Cannon, J. A. Zaia, D. L. DiGiusto, Genomic editing of the HIV-1 coreceptor CCR5 in adult hematopoietic stem and progenitor cells using zinc finger nucleases, Mol Ther 21, 1259-1269 (2013).
28. C. Mussolino, R. Morbitzer, F. Liitge, N. Dannemann, T. Lahaye, T. Cathomen, A novel TALE nuclease scaffold enables high genome editing activity in combination with low toxicity, Nucleic Acids Res. 39, 9283-9293 (2011).
29. L. Ye, J. Wang, A. I. Beyer, F. Teque, T. J. Cradick, Z. Qi, J. C. Chang, G. Bao, M. O. Muench, J. Yu, J. A. Levy, Y. W. Kan, Seamless modification of wild-type induced pluripotent stem cells to the natural CCR5Δ32 mutation confers resistance to HIV infection, Proc Natl Acad Sci USA 111, 9591-9596 (2014).
30. K. Newick, E. Moon, S. M. Albelda, Chimeric antigen receptor T-cell therapy for solid tumors, Molecular Therapy—Oncolytics 3, 16006 (2016).
31. H. E. Ghoneim, A. E. Zamora, P. G. Thomas, B. A. Youngblood, Cell-Intrinsic Barriers of T Cell-Based Immunotherapy, Trends Mol Med 22, 1000-1011 (2016).
32. J. J. Scholler, T. L. T. Brady, G. G. Binder-Scholl, W.-T. W. Hwang, G. G. Plesa, K. M. K. Hege, A. N. A. Vogel, M. M. Kalos, J. L. J. Riley, S. G. S. Deeks, R. T. R. Mitsuyasu, W. B. W. Bernstein, N. E. N. Aronson, B. L. B. Levine, F. D. F. Bushman, C. H. C. June, Decade-long safety and function of retroviral-modified chimeric antigen receptor T cells, Science Translational Medicine 4, 132ra53-132ra53 (2012).
33. M. Guadalupe, E. Reay, S. Sankaran, T. Prindiville, J. Flamm, A. McNeil, S. Dandekar, Severe CD4+ T-cell depletion in gut lymphoid tissue during primary human immunodeficiency virus type 1 infection and substantial delay in restoration following highly active antiretroviral therapy, J. Virol. 77, 11708-11717 (2003).
34. G. Dotti, S. Gottschalk, B. Savoldo, M. K. Brenner, Design and development of therapies using chimeric antigen receptor-expressing T cells, Immunol Rev 257, 107-126 (2014).
35. D. L. Hermanson, D. S. Kaufman, Utilizing chimeric antigen receptors to direct natural killer cell activity, Front Immunol 6, 195 (2015).
36. G. D. Trobridge, B. C. Beard, C. Gooch, M. Wohlfahrt, P. Olsen, J. Fletcher, P. Malik, H.-P. Kiem, Efficient transduction of pigtailed macaque hematopoietic repopulating cells with HIV-based lentiviral vectors, Blood 111, 5537-5543 (2008).
37. WO 2015/017755

All scientific and technical terms used in this application have meanings commonly used in the art unless otherwise specified.

As used herein, the term “subject” includes humans and non-human subjects. The term “non-human subject” includes all vertebrates, e.g., mammals and non-mammals, such as non-human primates, horses, sheep, dogs, cows, pigs, chickens, and other veterinary subjects and test subjects.

The use of the singular can include the plural unless specifically stated otherwise. As used in the specification and the appended claims, the singular forms “a”, “an”, and “the” can include plural referents unless the context clearly dictates otherwise. As used herein, “and/or” means “and” or “or”. For example, “A and/or B” means “A, B, or both A and B” and “A, B, C, and/or D” means “A, B, C, D, or a combination thereof” and said “combination thereof” means any subset of A, B, C, and D, for example, a single member subset (e.g., A or B or C or D), a two-member subset (e.g., A and B; A and C; etc.), or a three-member subset (e.g., A, B, and C; or A, B, and D; etc.), or all four members (e.g., A, B, C, and D).

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of treating, reducing, or inhibiting an infection by an immunodeficiency virus in a subject, which comprises transplanting one or more cells that express chimeric antigen receptors comprising an inhibitor of immunodeficiency virus fusion and human CD4 extracellular and transmembrane domains linked to a human CD3ζ signaling domain, in the subject, wherein expression of the inhibitor of immunodeficiency virus fusion is regulated by a ubiquitin C promoter and/or expression of the human CD4 extracellular and transmembrane domains linked to a human CD3ζ signaling domain is regulated by an EF-1 alpha promoter.

2. An expression vector comprising a nucleic acid sequence encoding an inhibitor of immunodeficiency virus fusion and human CD4 extracellular and transmembrane domains linked to a human CD3ζ signaling domain.

3. The expression vector according to claim 2, wherein the inhibitor of immunodeficiency virus fusion is C46.

4. The expression vector according to claim 2, wherein expression of the inhibitor of immunodeficiency virus fusion is regulated by a ubiquitin C promoter and/or expression of the human CD4 extracellular and transmembrane domains linked to a human CD3ζ signaling domain is regulated by an EF-1 alpha promoter.

5. The expression vector according to claim 2, wherein the expression vector is a lentiviral vector.

6. A host cell comprising an expression vector according to claim 2.

7. The host cell according to claim 6, wherein the host cell is a hematopoietic stem cell or a hematopoietic progenitor cell.

8. A cell that is the progeny of the host cell of claim 6.

9. The cell according to claim 6, wherein the cell expresses a chimeric antigen receptor encoded by the expression vector.

10. The cell according to claim 6, wherein the cell is a T cell, an NK cell, a B cell, or a tissue cell.

11. A method of treating, reducing, or inhibiting an infection by an immunodeficiency virus in a subject, which comprises transplanting one or more cells according to claim 6, in the subject.

12. The method according to claim 1, which further comprises administering to the subject a therapeutically effective amount of an antiretroviral therapeutic.

13. The method according to claim 1, which further comprises administering to the subject an effective amount of one or more latency reversing agents.

14. The method according to claim 1, which further comprises expanding the amount of cells expressing the chimeric antigen receptor in the subject by administering to the subject an effective amount of an agent that binds human CD4.

15. The method according to claim 14, wherein the agent is one or more antigens that bind human CD4.

16. The method according to claim 15, wherein the one or more antigens is gp120 or an epitope thereof.

17. The method according to claim 14, wherein the agent is an HIV vaccine that acts by binding human CD4.

18. The method according to claim 1, wherein the subject is human.

19. The expression vector according to claim 1, wherein the immunodeficiency virus is a human immunodeficiency virus, or a simian immunodeficiency virus having an envelope of the human immunodeficiency virus.

20. The expression vector according to claim 19, wherein the human immunodeficiency virus is HIV-1 or HIV-2.
